(19) United States
(12) Patent Application Publication
(10) Pub. No.: US 2023/0364210 A1
(43) Pub. Date: Nov. 16, 2023

(54) ß-AMYLOID VACCINE FOR THE TREATMENT OF ALZHEIMER’S DISEASE

(71) Applicant: OTHAIR PROTHENA LIMITED, Dublin 2 (IE)

(72) Inventors: Robin Barbour, Walnut Creek, CA (US); Gene Kinney, Boca Raton, FL (US); Wagner Zago, San Carlos, CA (US)

(21) Appl. No.: 18/245,523

(22) PCT Filed: May 19, 2021
(86) PCT No.: PCT/US2021/033180

§ 371 Date: Mar. 15, 2023

Related U.S. Application Data
(60) Provisional application No. 63/079,806, filed on Sep. 17, 2020.

(51) Int. Cl. A61K 39/00; C07K 7/06; C07K 7/08; A61P 25/28

(52) U.S. Cl. CPC A61K 39/0007; A61P 25/28; C07K 7/06; C07K 7/08; A61K 2039/545; A61K 2039/55555; A61K 2039/55572; A61K 2039/55577; A61K 2039/575; A61K 2039/6037

(57) ABSTRACT

The disclosure provides peptide compositions and immunotherapy compositions comprising an amyloid-beta (Aβ) peptide. The disclosure also provides methods of treating or effecting prophylaxis of Alzheimer’s disease or other diseases with beta-amyloid deposition in a subject, including methods of clearing deposits, inhibiting or reducing aggregation of Aβ, blocking the uptake by neurons, and clearing amyloid in a subject having or at risk of developing Alzheimer’s disease or other diseases containing amyloid-beta accumulations. The methods include administering to such patients the compositions comprising an. amyloid-beta (Aβ) peptide.

### RELATED APPLICATION

This application claims the benefit of U.S. Provisional Pat. Application No. 63/079,806, filed Sep. 17, 2020, which is incorporated by reference herein in its entirety.

### SEQUENCE LISTING STATEMENT

A computer readable form of the Sequence Listing is filed with this application by electronic submission and is incorporated into this application by reference in its entirety. The Sequence Listing is contained in the file created on May 18, 2021, having the file name “20-1083-WQ2_Sequence-Listing_ST25.txt” and is 18 kb in size.

### FIELD

The disclosure relates to the technical fields of immunology and medicine, and in particular to the treatment of Alzheimer’s disease and other diseases of protein misfolding,

### BACKGROUND

Alzheimer’s disease (AD) is a progressive disease resulting in senile dementia. Broadly speaking, the disease falls into two categories: late onset, which occurs in old age (65+years) and early onset, which develops well before the senile period, i.e., between 35 and 60 years. In both types of disease, the pathology is the same but the abnormalities tend to be more severe and widespread in cases beginning at an earlier age. The disease is characterized by at least two types of lesions in the brain, neurofibrillary tangles and senile plaques. Senile plaques (i.e., amyloid plaques) are areas of disorganized neuropil up to 150 µm across with extracellular amyloid deposits at the center which are visible by microscopic analysis of sections of brain tissue. The accumulation of amyloid plaques within the central nervous system is also associated with Down’s syndrome and other cognitive disorders, Cerebral amyloid angiopathy (CAA), and the ocular disease Age-Related Macular Degeneration,

A principal constituent of the plaques is a peptide termed Aβ or β-amyloid peptide. Aβ peptide is a 4-kDa internal fragment of 38-43 amino acids of a larger transmembrane glycoprotein named amyloid precursor protein (APP). As a result of proteolytic processing of APP by different secretase enzymes, Aβ is primarily found in both a short form, 40 amino acids in length, and a long form, ranging from 42-43 amino acids in length. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The cognitive and physical symptoms associated with this type of neural deterioration characterize Alzheimer’s disease.

Accordingly, there exists the need for new therapies and reagents for the prevention or treatment of Alzheimer’s disease, in particular, therapies and reagents capable of causing an immune response to the Aβ present in patients.

### SUMMARY

In some embodiments, disclosure is directed to one or more peptides comprising 3-10 amino acids from residues 1-10 or residues 12-25 of SEQ ID NO:01. For example, the peptide may include an amino acid sequence selected from the group consisting of any one of SEQ ID NO:02 to SEQ ID NO:96. In some embodiments, the peptide is from residues 1-7 of SEQ ID NO:01 and optionally a C-terminal cysteine and, as an example, include any one of SEQ ID NO:05 to SEQ ID NO:09, SEQ ID NO:13 to SEQ ID NO:16, SEQ ID NO:20 to SEQ ID NO:22, SEQ ID NO:26, SEQ ID NO:27, or SEQ ID NO:31. In some embodiments, the disclosure is directed to a peptide from residues 2-8 of SEQ ID NO:01 and optionally a C-terminal cysteine that, for example, include any one of SEQ ID NO:12 to SEQ ID NO:16, SEQ ID NO: 19 to SEQ ID NO:22, SEQ ID NO:25 to SEQ ID NO:27, SEQ ID NO:30, SEQ ID NO:31 or SEQ ID NO:34. In some embodiments, the disclosure is directed to a peptide from residues 12-24 or from residues 12-23 or from residues 12-22 or from residues 13-25 or from residues 13-24 or from residues 13-23 or from residues 13-22 or from residues 14-25 or from residues 14-24 or from residues 14-23 or from residues 14-22 or from residues 15-25 or from residues 15-24 or from residues 15-23 or from residues 15-22 of SEQ ID NO:01. In each of the embodiments, the peptide may include a C-terminal cysteine

in some embodiments, the disclosure is directed to a peptide of structure: [first peptide]-[linker 1 ]-[second peptide)-[linker 2]-[Cys], where the first peptide and the second peptide are the same or different and include, e.g., 3-10 amino acids from residues 1-10, 3-10 amino acids from residues 12-25 of SEQ ID NO:01, SEQ ID NO:02 through SEQ ID NO:96, and like sequences with an -RR dipeptide sequence appended to an end (e.g, SEQ ID NO: 101). In addition, linker 1 and linker 2 may be the same or different

In some embodiments, the peptide may include a linker, for example to a carrier, at a C-terminal portion of the peptide, which may include an amino acid sequence of AA, AAA, KK, KKK, SS, SSS, AGAG (SEQ ID NO:99), GG, GGG, GAGA(SEQ ID NO:98) and KGKG (SEQ ID NO:100). In some embodiments, the linker to the carrier, if present, may include a C-terminal cysteine (C). For example, the polypeptide may include the amino acid sequence of DAEFRHD-XXC (SEQ ID NO:05) or DAEFRHDRR-XXC (SEQ ID NO: 101), wherein XX and C are independently optional and, if present, XX can be AA, AAA, KK, KKK, SS, SSS AGAG (SEQ ID NO:99), GG, GGG, GAGA (SEQ ID NO:98) and KGKG (SEQ ID NO: 100) . In some embodiments, the peptide further comprises a blocked amine at the N-terminus.

In other embodiments, the disclosure is directed to an immunotherapy composition including the polypeptides of the disclosure, wherein the polypeptide may be linked to a carrier. The carrier may include serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

In other embodiments, the disclosure are directed to a pharmaceutical composition that includes the polypeptides and/or the immunotherapy compositions of the disclosure, and including at least one adjuvant. The adjuvant may be aluminum hydroxide, aluminum phosphate, aluminum sulfate; 3 De-O-acylated monophosphoryl lipid A (MPL) and synthetic analogs thereof, QS-21., QS-18, 08-17, QS-7, TQL1035, Complete Freund’s Adjuvant (CFA), Incomplete Freund’s Adjuvant (IFA), oil in water emulsions (such as squalene or peanut oil), CpG, polyglutamic acid, polylysine; AddaVax™, MF5911l, and combinations thereof. In addition, the formulation may include one or more of a liposomal formulation, a diluent, or a multiple antigen presenting system (MAP). The MAP may include one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

Embodiments of the disclosure are also directed to nucleic acid sequences encoding the polypeptides and the immunotherapy compositions of the disclosure. The nucleic acids may be included in a nucleic acid immunotherapy composition including the nucleic acid and at least one adjuvant.

In some embodiments, the disclosure is directed to a methods for treating or effecting prophylaxis of Alzheimers disease in a subject, and methods for inhibiting or reducing aggregation of Aft in a subject having or at risk of developing Alzheimer’s disease. The methods include administrating to the subject an immunotherapy composition, a nucleic acids immunotherapy composition, or a pharmaceutical formulation of the disclosure.

The methods of the disclosure may include repeating the administering at least a second time, at least a third time, at least a fourth time, at least a fifth time, or at least a sixth time, and may include repeating the administering at an interval of about bimonthly, of about 21 to about 28 days, of about quarterly, of about biannually, or of about annually.

Still further, methods of the disclosure are directed to inducing an immune response in an animal, The methods include administering to the animal a polypeptide, an immunotherapy composition, a pharmaceutical formulation or a nucleic acid immunotherapy composition of the disclosure in a regimen effective to generate an immune response including antibodies that specifically bind to Aβ. The immune response may include antibodies that specifically bind to the N-terminal region of Aβ.

In other embodiments, the disclosure is directed to an immunization kit including an immunotherapy composition of the disclosure and may include an adjuvant, wherein the immunotherapy composition may be in a first container and the adjuvant may be a second container.

Still further, the disclosure is directed to a kit including a nucleic acid immunotherapy composition of the disclosure and may include an adjuvant. The nucleic acid may be in a first container and the adjuvant may be in a second container.

### BRIEF DESCRIPTION OF THE FIGURES

FIG. 1 shows the results of an experiment comparing the titers of Guinea pig serum for amyloid beta single peptide immunogens QKLVFFAEC (SEQ ID NO.-40) and DAEFRHDC (SEQ ID NO:39). All immunogens comprised a C-terminal cysteine for coupling to maleimide activated CRM197 carrier. QS21 was utilized as an adjuvant in AddaVax squalene-based oil-in-water nano-emulsion.

FIG. 2 shows the results of an experiment measuring the titer of murine serum for amyloid beta single peptide immunogen AEFRHDSGC (SEQ ID NO:38) and DAEFRHDC (SEQ ID NO:39). The peptides were coupled to maleimide activated CRM197 carrier through the N-terminal cysteine. QS21 was used as an adjuvant.

### DESCRIPTION

The disclosure provides peptide compositions and immunotherapy compositions comprising an amyloid-beta (Aβ) peptide, The disclosure also provides methods of treating or effecting prophylaxis of Alzheimer’s disease or other diseases with beta-amyloid deposition in a subject, including methods of clearing and preventing formation of deposits, inhibiting or reducing aggregation of Aβ, blocking the binding and/or uptake of Aβ by neurons, inhibiting transmission of Aβ species between cells, and inhibiting propagation of pathology between brain regions in a subject having or at risk of developing Alzheimer’s disease or other diseases containing amyloid-beta accumulations. The methods include administering to such patients the compositions comprising an amyloid-beta (Aβ) peptide.

A number of terms are defined below. As used herein, the singular forms “a,” “an”, and “the” include plural referents unless the context clearly dictates otherwise. For example, the term “a compound” or “at least one compound” can include a plurality of compounds, including mixtures thereof.

Unless otherwise apparent from the context, the term “about” encompasses insubstantial variations, such as values within a standard margin of error of measurement (e.g., SEM) of a stated value. For example, the term “about” as used herein when referring to a measurable value such as a parameter, an amount, a temporal duration, can encompass variations of +/-10% or less, +/-5% or less, or +/-1 % or less or less of and from the specified value. Designation of a range of values includes all integers within or defining the range, and all subranges defined by integers within the range. As used herein, statistical significance means p<0.05,

Compositions or methods “comprising” or “including” one or more recited elements may include other elements not specifically recited. For example, a composition that “comprises” or “includes” a polypeptide sequence may contain the sequence alone or in combination with other sequences or ingredients.

An individual is at increased risk of a disease if the subject has at least one known risk-factor (e.g., age, genetic, biochemical, family history, and situational exposure) placing individuals with that risk factor at a statistically significant greater risk of developing the disease than individuals without the risk factor.

The term “patient” includes human and other mammalian subjects that receive either prophylactic or therapeutic treatment, including treatment naive subjects. As used herein, the terms “subject” or “patient” refer to any single subject for which treatment is desired, including other mammalian subjects such as, humans, cattle, dogs, guinea pigs, rabbits, and so on. Also intended to be included as a subject are any subjects involved in clinical research trials not showing any clinical sign of disease, or subjects involved in epidemiological studies, or subjects used as controls.

The term “disease” refers to any abnormal condition that impairs physiological function. The term is used broadly to encompass any disorder, illness, abnormality, pathology, sickness, condition, or syndrome in which physiological function is impaired, irrespective of the nature of the etiology.

The term “symptom” refers to a subjective evidence of a disease, such as altered gait, as perceived by the subject. A “sign” refers to objective evidence of a disease as observed by a physician.

As used herein, the terms “treat” and “treatment” refer to the alleviation or amelioration of one or more symptoms or effects associated with the disease, prevention, inhibition or delay of the onset of one or more symptoms or effects of the disease, lessening of the severity or frequency of one or more symptoms or effects of the disease, and/or increasing or trending toward desired outcomes as described herein.

The terms “prevention”, “prevent”, or “preventing” as used herein refer to contacting (for example, administering) the peptide(s) or immunotherapy compositions of the present disclosure with a subject before the onset of a disease, with or without Aβ pathology already present (primary and secondary prevention), thereby delaying the onset of clinical symptoms and/or alleviating symptoms of the disease after the onset of the disease, compared to when the subject is not contacted with the peptide or immunotherapy compositions, and does not refer to completely suppressing the onset of the disease. In some cases, prevention may occur for limited time after administration of the peptide or immunotherapy compositions of the present disclosure. In other cases, prevention may occur for the duration of a treatment regimen comprising administering the peptide or immunotherapy compositions of the present disclosure.

The terms “reduction”, “reduce”, or “reducing” as used herein refer to decreasing the amount of Aβ and/or tau present in a subject or in tissue of the subject, or suppressing an increase in the amount of Aβ present in a subject or tissue in a subject, which encompasses decreasing or suppressing an increase in (e.g, decreasing the rate of increase) the amount of Aβ present, accumulated, aggregated, or deposited in the subject or tissue in the subject. In certain embodiments, the decrease in or suppression of an increase in (e.g., decreasing the rate of increase) the amount of Aβ present, accumulated, aggregated, or deposited in the subject refers to an amount of Aβ present, accumulated, aggregated, or deposited in the central nervous system (CNS) of the subject. In certain embodiments, the decrease in or suppression of an increase in (e.g. decreasing the rate of increase) the amount of Aβ present; accumulated, aggregated, or deposited in the subject refers to an amount of Aβ present, accumulated, aggregated, or deposited in the periphery (e.g., peripheral circulatory system) of the subject. In certain embodiments, the decrease in or suppression of an increase in (e.g, decreasing the rate of increase) the amount of Aβ present, accumulated, aggregated, or deposited in the subject refers to an amount of Aβ present, accumulated, aggregated, or deposited in the brain of the subject. In some embodiments, the Aβ reduced is the pathological form(s) of the Aβ (e.g. extracellular plaque deposits of the β-amyloid peptide (Aβ); neuritic amyloid plaques). In yet other embodiment, pathological indicators of neurodegenerative disease and/or β-amyloidopathies are decreased.

The terms “epitope” or “antigenic determinant” refers to a site on an antigen to which B and/or T cells respond, or to a site on an antigen to which an antibody binds. Epitopes can be formed both from contiguous amino acids or from noncontiguous amino acids jaxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, at least 4, at least 5, at least 6, at least 7, at least 8, at least 9 or at least to amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed. (1996).

An “immunogenic agent” or “immunogen” or “antigen” is capable of inducing an immunological response against itself or modified/processed versions of itself upon administration to an animal, optionally in conjunction with an adjuvant. The terms “immunogenic agent” or “immunogen” or “antigen” refer to a compound or composition comprising a peptide, polypeptide or protein which is “antigenic” or “immunogenic” when administered in an appropriate amount (an “immunogenically effective amount”),. i.e., capable of inducing, eliciting, augmenting or boosting a cellular and/or humoral immune response and of being recognized by the products of that response (T cells, antibodies). An immunogen can be a peptide, or a combination of two or more peptides, that includes at least 3, at least 4, at least 5, at least 6, at least 7, at least 8, at least 9, or at least 10 amino acids in a liner or spatial conformation. An immunogen may be effective when given alone or in coinbination, or linked to, or fused to, another substance (which can be administered at one time or over several intervals). An immunogenic agent or immunogen may include an antigenic peptide or polypeptide that is linked to a carrier as described herein.

A nucleic acid such as DNA or RNA that encodes an antigenic peptide or polypeptide is referred to as a “DNA [or RNA] immunogen,” as the encoded polypeptide is expressed in vivo after administration of the DNA or RNA. The peptide or polypeptide can be recombinantly expressed from a vaccine vector, which can be naked DNA or RNA that comprises the peptide or polypeptide coding sequence operably linked to a promoter, e.g., an expression vector or cassette as described herein.

The term “adjuvant” refers to a compound that, when administered in conjunction with an antigen, augments the immune response to the antigen, but when administered alone does not generate an immune response to the antigen. Adjuvants can augment an immune response by several mechanisms including lymphocyte recruitment, stimulation of B and/or T cells, and stimulation of macrophages. An adjuvant may be a natural compound, a modified version of or derivative of a natural compound, or a synthetic compound.

The terms “peptide” and “polypeptide” are used interchangeably herein and refer to a chain of two or more consecutive amino acids. If and when a distinction is made, context makes the meaning clear. For example, if two or more peptides described herein are joined to make a dimeric or multimeric peptide, polypeptide may be used to indicate “poly” or “more than one” peptide.

The term “pharmaceutically acceptable” means that the carrier, diluent, excipient, adjavant, or auxiliary is compatible with the other ingredients of a pharmaceutical formulation and not substantially deleterious to the recipient thereof.

The terms “immunotherapy” or “immune response” refer to the development of a beneficial humoral (antibody mediated) and/or a cellular (mediated by antigen-specific T cells or their secretion products) response directed against an Aβ peptide in a recipient. Such a response can be an active response induced by administration of immunogen (e.g. an Aβ peptide). A cellular immune response is elicited by the presentation of polypeptide epitopes in association with Class I or Class II MHC molecules to activate antigen-specific CD4⁺ T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T-cells from an immunized syngeneic animal and measuring protective or therapeutic effect in a second subject

### Amyloid Beta (Aβ)

AI) (also referred to herein as beta amyloid peptide or Abeta) peptide is about a 4-kDa internal fragment of 38-43 amino acids of APP (Aβ39, Aβ40, Aβ41, Aβ42, and Aβ43). Aβ40, for example, consists of residues 672-711 of APP and Aβ42 consists of residues 673-713 of APP. As a result of proteolytic processing of APP by different secretase enzymes in vivo or in situ, Aβ is found in both a “short form”, 40 amino acids in length, and a “long form”, ranging from 42-43 amino acids in length. Epitopes or antigenic determinants, as described herein, are located within the N-terminus of the Aβ peptide and include residues within amino acids 1-10 of Aβ, for example from residues 1-3, 14, 1-5, 1-6, 1-7, or 3-7 of Aβ42. Additional examples of epitopes or antigenic determinants include residues 2-4, 2-5, 2-6, 2-7, or 2-8 of Aβ, residues 3-5, 3-6, 3-7, 3-8, or 3-9 of Aβ, or residues 4-7, 4-8, 4-9, or 4-10 of Aβ42. Epitopes or antigenic determinants, as described herein, are also located in a central region of the Aβ peptide and include residues within amino acid residues 12-25, within residues 12-24, within residues 12-23, within residues 12-22, within residues 13-25, within residues 13-24, within residues 13-23, within residues 13-22, within residues 14-25, within residues 14-24, within residues 14-23, within residues 14-22, within residues 15-25, within residues 15-24, within residues 15-23, or within residues 15-22 of Aβ. For example, from residues 12-17, 12-18, 12-19, 12-20, 12-21, 13-17, 13-18, 13-19, 13-20, 13-21, 13-22, 14-17, 14-18, 14-19, 14-20, 14-21, 14-22, 14-23, 15-17, 15-18, 15-19, 15-20, 15-21, 15-22, 15-23, or 15-24 of Aβ42. Additional examples of epitopes or antigenic determinants include residues 16-18, 16-19, 16-20, 16-21, 16-22, 16-23, 16-24, 16-25, 17-19, 17-20, 17-21, 17-22, 17-23, 1 7-24 or 17-25 of Aβ42, Other examples of epitopes or antigenic determinants include residues 18-20, 18-21, 18-22, 18-23, 18-24, 18-25, 19--21.. 19-22, 19--23, 19-24, 19-25, 20-22, 20-23, 20-24, 20-25, 21-23, 21-24 or 21-25 of Aβ42. Aβ is the principal component of characteristicplaques of Alzheimer’s disease. Aβ is generated by processing of a larger protein APP by two enzymes, termed beta and gamma secretases, Known mutations in APP associated with Alzheimer’s disease occur proximate to the site of beta or gamma secretase, or within Aβ. Part of the hydrophobic transmembrane domain of APP is found at the carboxy end of Aβ, and may account for the ability of Aβ to aggregate into plaques, particularly in the case of the long form. Accumulation of amyloid plaques in the brain eventually leads to neuronal cell death. The physical symptoms associated with this type of neural deterioration characterize Alzheimer’s disease.

### Peptide Immunogens

An agent used for active immunization can induce in a patient an immune response and can serve as an imumnotherapy. Agents used for active immunization can be, for example, the same types of immunogens used for generating monoclonal antibodies in laboratory animals, and may include 3, 4, 5, 6, 7, 8, 9, 10, 11, or 12 or more contiguous amino acids from a region of Aβ peptide.

In some embodiments of the disclosure, the immunogen can include an Aβ peptide comprising 3-10 amino acids from residues 1-10 of the N-terminal sequence of Aβ (SEQ ID NO:01). In some embodiments of the disclosure, the immunogen can include an Aβ peptide comprising 3-10 amino acids from residues 12-25 of Aβ (SEQ ID NO:01), In some embodiments, the peptide is unphosphorylated. In some embodiments, the peptide is phosphorylated at serine (S), threonine (T), and/or tyrosine (Y) phosphorylation sites. In each of the embodiments of the peptide described herein, the peptide may comprise, consist, or consist essentially of the recited sequences.

In some embodiments of the disclosure, the Aβ peptide immunogen can include 3-10 amino acids from residues 1-10 or residues 12-25 of DAEFRHDSGYEVHHQKLVFFAEDVGSNKGAIIGLMVGGVVIA (SEQ ID NO:01). For example, the Aβ peptide includes the following:

| | |
|---|---|
| DAEFRHDSGY | (SEQ ID NO:02) |
| DAEFRHDSG | (SEQ ID NO:03) |
| DAEFRHDS | (SEQ ID NO:04) |
| DAEFRHD | (SEQ ID NO:05) |
| DAEFRH | (SEQ ID NO:06) |
| DAEFR | (SEQ ID NO:07) |
| DAEF | (SEQ ID NO:08) |
| DAE | (SEQ ID NO:09) |
| AEFRHDSGY | (SEQ ID NO:10) |
| AEFRHDSG | (SEQ ID NO:11) |
| AEFRHDS | (SEQ ID NO:12) |
| AEFRHD | (SEQ ID NO:13) |
| AEFRH | (SEQ ID NO:14) |
| AEFR | (SEQ ID NO:15) |
| AEF | (SEQ ID NO:16) |
| EFRHDSGY | (SEQ ID NO:17) |
| EFRHDSG | (SEQ ID NO:18) |
| EFRHDS | (SEQ ID NO:19) |
| EFRHD | (SEQ ID NO:20) |
| EFRH | (SEQ ID NO:21) |
| EFR | (SEQ ID NO:22) |
| FRHDSGY | (SEQ ID NO:23) |
| FRHDSG | (SEQ ID NO:24) |
| FRHDS | (SEQ ID NO:25) |
| FRHD | (SEQ ID NO:26) |
| FRH | (SEQ ID NO:27) |
| RHDSGY | (SEQ ID NO:28) |
| RHDSG | (SEQ ID NO:29) |
| RHDS | (SEQ ID NO:30) |
| RHD | (SEQ ID NO:31) |
| HDSGY | (SEQ ID NO:32) |
| HDSG | (SEQ ID NO:33) |
| HDS | (SEQ ID NO:34) |
| DSGY | (SEQ ID NO:35) |
| DSG | (SEQ ID NO:36) |
| SGY | (SEQ ID NO:37) |
| AEFRHDSGC | (SEQ ID NO:38) |
| DAEFRHDC | (SEQ ID NO:39) |
| QKLVFFAEC | (SEQ ID NO:40) |
| VHHQKLVFFA | (SEQ ID NO:41) |
| VHHQKLVFF | (SEQ ID NO:42) |
| VHHQKLVF | (SEQ ID NO:43) |
| VHHQKLV | (SEQ ID NO:44) |
| VHHQKL | (SEQ ID NO:45) |
| HHQKLVFFAE | (SEQ ID NO:46) |
| HHQKLVFFA | (SEQ ID NO:47) |
| HHQKLVFF | (SEQ ID NO:48) |
| HHQKLVF | (SEQ ID NO:49) |
| HHQKLV | (SEQ ID NO:50) |
| HHQKL | (SEQ ID NO:51) |
| HQKLVFFAED | (SEQ ID NO:52) |
| HQKLVFFAE | (SEQ ID NO:53) |
| HQKLVFFA | (SEQ ID NO:54) |
| HQKLVFF | (SEQ ID NO:55) |
| HQKLVF | (SEQ ID NO:56) |
| HQKLV | (SEQ ID NO:57) |
| HQKL | (SEQ ID NO:58) |
| QKLVFFAEDV | (SEQ ID NO:59) |
| QKLVFFAED | (SEQ ID NO:60) |
| QKLVFFAE | (SEQ ID NO:61) |
| QKLVFFA | (SEQ ID NO:62) |
| QKLVFF | (SEQ ID NO:63) |
| QKLVF | (SEQ ID NO:64) |
| QKLV | (SEQ ID NO:65) |
| QKL | (SEQ ID NO:66) |
| KLVFFAEDVG | (SEQ ID NO:67) |
| KLVFFAEDV | (SEQ ID NO:68) |
| KLVFFAED | (SEQ ID NO:69) |
| KLVFFAE | (SEQ ID NO:70) |
| KLVFFA | (SEQ ID NO:71) |
| KLVFF | (SEQ ID NO:72) |
| KLVF | (SEQ ID NO:73) |
| KLV | (SEQ ID NO:74) |
| LVFFAEDVG | (SEQ ID NO:75) |
| LVFFAEDV | (SEQ ID NO:76) |
| LVFFAED | (SEQ ID NO:77) |
| LVFFAE | (SEQ ID NO:78) |
| LVFFA | (SEQ ID NO:79) |
| LVFF | (SEQ ID NO:80) |
| LVF | (SEQ ID NO:81) |
| VFFAEDVG | (SEQ ID NO:82) |
| VFFAEDV | (SEQ ID NO:83) |
| VFFAED | (SEQ ID NO:84) |
| VFFAE | (SEQ ID NO:85) |
| VPFA | (SEQ ID NO:86) |
| VFF | (SEQ ID NO:87) |
| FFAEDVG | (SEQ ID NO:88) |
| FFAEDV | (SEQ ID NO:89) |
| FFAED | (SEQ ID NO:90) |
| FFAE | (SEQ ID NO:91) |
| FFA | (SEQ ID NO:92) |
| FAEDVG | (SEQ ID NO:93) |
| FAEDV | (SEQ ID NO:94) |
| FAED | (SEQ ID NO:95) |
| FAE | (SEQ ID NO:96) |
| DAEFRHDRR | (SEQ ID NO:101) |

In some embodiments, the Aβ peptide is DAEFRHD (SEQ ID NO:05), AEFRHDS (SEQ ID NO: 12), or EFRHDSG (SEQ ID NO:18). Each Aβ sequence optionally further comprises a C-terminal cysteine like, for example, AEFRHDSGC (SEQ ID NO:38), DAEFRHDC (SEQ ID NO:39) and QKLVFFAEC (SEQ ID NO:40). In each of these embodiments, the peptide may comprise, consist, or consist essentially of the recited sequences

In some embodiments, the Aβ peptide according to SEQ ID NO:1 through SEQ ID NO:96 further includes an arginine-arginine dipeptide (RR) at the N-terminal, the C-terminal or both terminii. For example, SEQ ID NO:101 (DAEFRHDRR) is comprised of DAEFRHD (SEQ ID NO:05) with an -RR dipeptide at the C-terminus.

In some embodiments, the immunogen as described herein further comprises a linker (for example, to a carrier) at a C-terminal portion or N-terminal portion of the polypeptide. In some embodiments, the linker comprises an amino acid sequence including AA, AAA, KK, KKK, SS, SSS, AGAG (SEQ ID NO:99), GG, GGG, GAGA (SEQ ID NO:98), and KGKG (SEQ ID NO:100). In some embodiments, the peptide further comprises a C-terminal cysteine, and some embodiments that comprise a linker further comprise a C-terminal cysteine on the C-terminal end of the linker. In some embodiments, the peptide further comprises a N-terminal cysteine, and some embodiments that comprise a linker further comprise a N-terminal cysteine on the N-terminal end of the linker. In some embodiments, the immunogen peptides further comprise a blocked amine at the N-terminus,

In some embodiments, the two or more Aβ peptides are linked to form an Aβ polypeptide. The one or more Aβ peptides can be linked by an intra-peptide linker(s), which linker is as described above and herein. For example, a polypeptide linker located between the C-terminal of the first peptide and the N terminal of the second peptide. With or without the intra-peptide linker, the Aβ polypeptide may be arranged in any order. For example, a specific Aβ peptide (“Aβ 1”) may be positioned at the N-terminal portion of a dual Aβ polypeptide and the same or a different Aβ peptide (for this example, a different Aβ, “Aβ 2”) may be positioned at the C-terminal portion of the dual polypeptide. Or, the Aβ peptides in this example could be arranged in the opposite orientation (Aβ 2 N-terminal to Aβ 1). Reference to a first peptide or a second peptide herein is not intended to suggest an order of the Aβ peptides in embodiments that comprise more than one Aβ peptide of the immunogens.

In addition, the either the N-terminal or C-terminal portion of the Aβ peptide or Aβ polypeptide can include a linker for conjugating the peptides or the polypeptide to a carrier, which linker is as described above and herein. In some embodiments, the Aβ peptide or polypeptide that comprise a linker further comprise a C-terminal cysteine on the C-terminal or N-terminal end of the linker. In some embodiments, the immunogen peptides further comprise a blocked amine at the N-terminus. In some embodiments, any of the Aβ peptides or polypeptides may include a C-terminal cysteine without the linker.

When the Aβ peptides are linked to form a Aβ polypeptide, the linker may be a cleavable linker. As used herein, the term “cleavable linker” refers to any linker between the antigenic peptides that promotes or otherwise renders the Aβ polypeptide more susceptible to separation from each other by cleavage (for example, by endopeptidases, proteases, low pH or any other means that may occur within or around the antigen-presenting cell) and, thereby, processing by the antigen-presenting cell, than equivalent peptides lacking such a cleavable linker. In some embodiments, the cleavable linker is a protease-sensitive dipeptide or oligopeptide cleavable linker. In certain embodiments, the cleavable linker is sensitive to cleavage by a protease of the trypsin family of proteases. In some embodiments, the cleavable linker comprises an amino acid sequence including arginine-arginine (Arg-Arg), arginine-arginine-valine-arginine (Arg-Val-Arg-Arg; SEQ ID NO:97), Gly-Ala-Gly-Ala (SEQ ID NO:98), Ala-Gly-Ala-Gly (SEQ ID NO:99), Lys-Gly-Lys-Gly (SEQ ID NO: 100), valine-citrulline (Val-Cit), valine-arginine (Val-Ars), valine-lysine (Val-Lys), valine-alanine (Val-Ala), and phenylalanine-lysine (Phe-Lys). In some embodiments, the cleavable linker is arginine-arginine (Arg-Arg).

In some embodiments, the linker comprises between about 1-10 amino acids, about 1-9 amino acids, about 1-8 amino acids, about 1-7 amino acids, about 1-6 amino acids, about 1-5 amino acids, about 1-4 amino acids, about 1-3 amino acids, about 2 amino acids or one (1) amino acid. In some embodiments, the linker is one amino acid, two amino acids, three amino acids, four amino acids, five amino acids, six amino acids, seven amino acids, eight amino acids, nine amino acids, or ten amino acids.

In some embodiments, the amino acid composition of a linker can mimic the composition of linkers found in natural multidomain proteins, where certain amino acids are overrepresented, underrepresented or equi-represented in natural linkers as compared to their abundance in whole protein. For example, threonine (Thr), serine (Ser), proline (Pro), glycine (Gly), aspartic acid (Asp), lysine (Lys), glutamine (Gln), asparagine (Asn), arginine (Arg), phenylalanine (Phe), glutamic acid (Glu) and alanine (Ala) are overrepresented in natural linkers. In contrast, isoleucine (He), tyrosine (Tyr), tryptophan (Trp), and cysteine (Cys) are underrepresented. In general, overrepresented amino acids were polar uncharged or charged residues, which constitute approximately 50% of naturally encoded amino acids, and Pro, Thr, and Gln were the most preferable amino acids for natural linkers. See, e.g., Chen, X. et al., “Fusion Protein Linkers: Property, Design and Functionality” Adv Drug Deliv Rev., 15; 65(10): 1357-1369 (2013).

In some embodiments, the amino acid composition of a linker can mimic the composition of linkers commonly found in recombinant proteins, which can generally by classified as flexible or rigid linkers. For example, flexible linkers found in recombinant proteins are generally composed of small, non-polar (e.g. Gly) or polar (e.g. Ser or Thr) amino acids whose small size provides flexibility and allows for mobility of the connecting functional domains. The incorporation of, e.g., Ser or Thr can maintain the stability of the linker in aqueous solutions by forming hydrogen bonds with the water molecules, and therefore can reduce interactions between the linker and the immunogens. In some embodiments, a linker comprises stretches of Gly and Ser residues (“GS” linker). An example of a widely used flexible linker is (Gly-Gly-Ser)n, (Gly-Gly-Gly-Ser)n or (Gly-Gly-Gly-Gly-Ser)n, where n=1-3. Adjusting the copy number “n” can optimize a linker to achieve sufficient separation of the functional immunogen domains to, e.g., maximize an immunogenic response. Many other flexible linkers have been designed for recombinant fusion proteins that can be used herein. In some embodiments, linkers can be rich in small or polar amino acids such as Gly and Ser but also contain additional amino acids such as Thr and Ala to maintain flexibility, as well as polar amino acids such as Lys and Glu to improve solubility. See, e.g., Chen, X. et al., Adv Drug Deliv Rev., 15: 65(10): 1357-1369 (2013).

In some embodiments of the disclosure, the Aβ polypeptide comprises, consists essentially of or consists of an amino acid sequence selected from DAEFRHD (SEQ ID NO:05), DAEFRHDRR (SEQ ID NO:101), EFRHDSG (SEQ ID NO:18), AEFRHDS (SEQ ID NO: 12), or QKLVFFAE (SEQ ID NO:61) wherein XX is optionally appended to the C-terminal end of SEQ ID NOS:05, 101, 12, or 18, and a cysteine is optionally appended to the C-terminal end of SEQ ID NOS:05, 101, 12, or 18, or if XX is present, to the C-terminal end of the XX. XX can be the same or different amino acids and in some embodiments are AA, AAA, KK, KKK, SS, SSS,, GG and GGG. In addition, XX may represent AGAG (SEQ ID NO:99), GAGA (SEQ ID NO:98), and KGKG (SEQ ID NO:100).

In some embodiments, the dual Aβ polypeptide is as follows, from N-terminal to C-terminal:
[first peptide]-[linker 1]-[second peptide]-[linker 2]-[Cys], or
[Cys]-[linker 1]-[first peptide]-[linker 2]-[second peptide],
wherein, the first peptide is a Aβ peptide and the second peptide is the same or different Aβ peptide, and each of linker 1, linker 2 and [Cys] is optional. In some embodiments, both the first peptide and the second peptide are from residues 1-10 of SEQ ID NO:01, and may be the same or different. In some embodiments, both of the first peptide or the second peptide are from residues 12-25 of SEQ ID NO:01, and may be the same or different. In some embodiments, either the first or the second peptide is from residues 1-10 of SEQ ID NO:01 and the other peptide is from residues 12-25 of SEQ ID NO:01. In some embodiments, linker 1 and linker 2 may be the same or different

In some embodiments, at least one of the first peptide or the second peptide is selected from SEQ ID NO:02 through SEQ ID NO-39, and in some embodiments, both the first peptide and the second peptide are selected from SEQ ID NO:02 through SEQ ID NO:39. In some embodiments, at least one of the first peptide or the second peptide is selected from SEQ ID NO:40 through SEQ ID NO:96, and in some embodiments, both the first peptide and the second peptide are selected from SEQ ID NO:40 through SEQ ID NO:96. In some embodiments, either the first or the second peptide is selected from SEQ ID NO:02 through SEQ ID NO:39 and SEQ ID NO.101, and the other peptide is selected from SEQ ID NO:40 through SEQ ID NO:96 and SEQ ID NO:101. In some embodiments, either the first peptide or the second peptide includes a peptide according to SEQ ID NO:02 through SEQ ID NO:96 further comprising an RR- or -RR at the N-terminal or C-terminal end; respectively. In some embodiments, both the first and the second peptide includes a peptide according to SEQ ID NO:02 through SEQ ID NO:96 further comprising an RR- or -RR dipeptide at the N-terminal or C-terminal end, respectively. In some embodiments, the -RR dipeptide is on the C-terminus.

In some embodiments, each of the first peptide and the second peptide include residues 1-10 of SEQ ID NO:01, or residues 12-25 of SEQ ID NO:01, or one of SEQ ID NO:02 through SEQ ID NO:96, or SEQ ID NO:101. or a peptide according to SEQ ID NO:02 through SEQ ID NO:96 further comprising an RR- or -RR at the N-terminal or C-terminal end, respectively.

Non-limiting examples of the Aβ peptides include SEQ ID NO:02 to SEQ ID NO:96, and like sequences further including a C-terminal cysteine or C-terminal -RR dipeptide, for example SEQ ID NO:101.

### Peptide-Carrier Immunogens

Aβ peptides (and polypeptides thereof) are immunogens in accordance with the disclosure. In some embodiments, the peptides described herein can be linked to a suitable carrier to help elicit an immune response. Accordingly, one or more the peptides and polypeptides of the disclosure can be linked to a carrier. For example, the Aβ peptide may be linked to the carrier with or without a linker as described above and herein and, optionally, a C-terminal cysteine at C-terminal end of the linker and, if a linker is absent, at the C-terminal end of the peptide. For example, each Aβ peptide or polypeptide may be linked to the carrier with or without spacer amino acids (e.g., Gly-Gly, Gly-Gly-Gly, Ala-Ala, Ala-Ala-Ala, Lys-Lys, Lys-Lys-Lys, Ser-Ser, Ser-Ser-Ser, Gly-Ala-Gly-Ala (SEQ ID NO:98), Ala-Gly-Ala-Gly (SEQ ID NO:99), or Lys-Gly-Lys-Gly (SEQ ID NO:100)) and optionally, a C-terminal cysteine or N-terminal cysteine to provide a linker between the peptide(s) and the carrier.

Suitable carriers include, but are not limited to serum albumins, keyhole limpet hemocyanin, immunoglobulin molecules, thyroglobulin, ovalbumin, tetanus toxoid, or a toxoid from other pathogenic bacteria, such as diphtheria (e.g., CRM197), E. coli, cholera, or H. pylori, or an attenuated toxin derivative. T cell epitopes are also suitable carrier molecules. Some conjugates can be formed by linking peptide immunogens of the invention to an immunostimulatory polymer molecule (e.g., tripalmitoyl-S-glycerine cysteine (Pam3Cys), mannan (a mannose polymer), or glucan (a β 1-2 polymer)), cytokines (e.g., IL-1, IL-1 alpha and β peptides, IL-2, γ-INF, IL-10, GM-CSF), and chemokines (e.g., MIP1-α and β, and RANTES). Additional carriers include virus-like particles. In some compositions, immunogenic peptides can also be linked to carriers by chemical crosslinking. Techniques for linking an immunogen to a carrier include the formation of disulfide linkages using N-succinimidyl-3-(2-pyridyl-thi-o)propionate (SPDP), and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine resides on one protein and an amide linkage through the epsilon-amino on a lysine, or other free amino group in other amino acids. In some embodiments, chemical crosslinking can comprise use of SBAP (succinimidyl 3-(bromoacetamido)propionate), which is a short (6.2 angstrom) cross-linker for amine-to-sulfhydiyl conjugation via N-hydroxysuccinimide (NHS) ester and bromoacetyl reactive groups. A variety of such disulfide/amide-forming agents are described by Jansen et al., “Immunotoxins: Hybrid Molecules Combining High Specificity and Potent Cytotoxicity” Immunological Reviews 62:185-2 16 (February 1982). Other bifunctional coupling agents form a thioether rather than a disulfide linkage. Many of these thio-ether-forming agents are commercially available and include reactive esters of 6-maleimidocaproic acid, 2-bromoacetic acid, and 2-iodoacetic acid, 4-(N-maleimido-methyl)cy- clohexane-1 -carboxylic acid. The carboxyl groups can be activated by combining them with succinimide or 1-hydroxyl-2-nitro-4-sulfonic acid, sodium salt, Virus-like particles (VLPs), also called pseudovirions or virus-derived particles, represent subunit structures composed of multiple copies of a viral capsid and/or envelope protein capable of self-assembly into VLPs of defined spherical symmetry in vivo, (Powilleit, et al, (2007) PLoS ONE 2(5):e41 5.) Alternatively, peptide immunogens can be linked to at least one artificial T-cell epitope capable of binding a large proportion of MHC Class 11 molecules, such as the pan DR epitope (“PADRE”). Pan DR-binding peptides (PADRE) are described in US 5,736,142, WO 95/07707, and Alexander, el al, Immunity, 1:751-761 (1994).

Active immunogens can be presented in multimeric form in which multiple copies of an immunogen are presented on a carrier as a single covalent molecule. In some embodiments, the carrier includes various forms of the Aβ peptide. For instance, the Aβ peptide of the immunogen can include peptides that have different Aβ antigens in different orders, or may be present with or without an intrapeptide linker and/or a linker to a carrier.

In some compositions, the immunogenic peptides can also be expressed as fusion proteins with carriers. In certain compositions, the immunogenic peptides can be linked at the amino terminus, the carboxyl terminus, or internally to the carrier. In some compositions, the carrier is CRM197. In some compositions, the carrier is diphtheria toxoid.

### Nucleic Acids

The disclosure further provides nucleic acids encoding any of the amyloid-beta (Aβ) peptides as disclosed herein. The nucleic acid immnunotherapy compositions as disclosed herein, comprise, consist essentially of, or consist of a nucleic acid sequence encoding one or more amyloid-beta (Aβ) peptides as disclosed herein. For example, the Aβ peptide includes a sequence that is 3-10 amino acid residues in length and from the first ten N-terminal residues of SEQ ID NO:01 or from residues 12-25 of SEQ ID NO:01. Accordingly. and as a non-limiting example, one or more nucleic acids encoding any of SEQ ID NOS:2-96 and 101 provide an immunogen and a component of a pharmaceutical composition of the disclosure. Likewise, one or more nucleic acids may encode any of SEQ ID NOS:2-96 with an RR- N-terminal or -RR C-terminal dipeptide. In certain embodiments, the peptide sequences may be encoded by the some or separate nucleic acid sequences that may also encode a linker to the carrier and an N- or C-terminal cysteine as described herein. In addition, when a single nucleic acid sequence encodes more than one Aβ peptide, the sequence may also encode a linker or cleavable linker as described herein.

A nucleic acid such as DNA that encodes an immunogen and is used as a vaccine can be referred to as a “DNA immunogen” or “DNA vaccine” as the encoded polypeptides are expressed in vivo after administration of the DNA. DNA vaccines are intended to induce antibodies against the proteins of interest they encode in a subject by: integrating DNA encoding the proteins of interest into a vector (a plasmid or virus); administering the vector to the subject; and expressing the proteins of interest in the subject in which the vector has been administered to stimulate the immune system of the subject. A DNA vaccine remains in the body of the subject for a long time after the administration, and continues to slowly produce the encoded proteins. Thus, excessive immune responses can be avoided. DNA vaccines can also be modified using a genetic engineering techniques. Optionally, such nucleic acids further encode a signal peptide and can be expressed with the signal peptide linked to peptide, Coding sequences of nucleic acids can be operably linked with regulatory sequences to ensure expression of the coding sequences, such as a promoter, enhancer, ribosome binding site, transcription termination signal, and the like. The nucleic acids encoding Aβ can occur in isolated form or can be cloned into one or more vectors. The nucleic acids can be synthesized by, for example, solid state synthesis or PCR of overlapping oligonucleotides. Nucleic acids encoding Aβ peptide or Aβ polypeptides with and without linkers and/or cleavable linkers and with or without protein-based carriers can be joined as one contiguous nucleic acid, e.g.. within an expression vector.

DNA is more stable than RNA, but involves some potential safety risks such as induction of anti-DNA antibodies, thus in some embodiments, the nucleic acid can be RNA. RNA nucleic acid that encodes an immunogen and is used as a vaccine can be referred to as a “RNA immunogen” or “RNA vaccine” or “mRNA vaccine” as the encoded polypeptides are expressed in vivo after administration of the RNA. Ribonucleic acid (RNA) vaccines can safely direct a subject’s cellular machinery to produce one or more polypeptide(s) of interest. In some embodiments, a RNA vaccine can be a non-replicating mRNA (massenger-RNA) or a virally derived, self-amplifying RNA. mRNA-based vaccines encode the antigens of interest and contain 5′ and 3′ untranslated regions (UTRs), whereas self-amplifying RNAs encode not only the antigens, but also the viral replication machinery that enables intracellular RNA amplification and abundant protein expression. In vitro transcribed mRNA can be produced from a linear DNA template using a T7, a T3 or an Sp6 phage RNA polymerase. The resulting product can contain an open reading frame that encodes the peptides of interest as disclosed herein, flanking 5′- and 3′-UTR sequences, a 5′ cap and a poly(A) tail. In some embodiments, a RNA vaccine can comprise trans-amplifying RNA (for example, see Beissert et al., Molecular Therapy January 2020 28(1):119-128). In certain embodiments, RNA vaccines encode an Aβ peptide and a tau peptide as disclosed herein, and are capable of expressing the Aβ and a tau peptides, in particular if transferred into a cell such as an immature antigen presenting cell. RNA may also contain sequences which encode other polypeptide sequences such as immune stimulating elements, In some embodiments, the RNA of a RNA vaccine can be modified RNA. The term “modified” in the context of the RNA can include any modification of RNA which is not naturally present in RNA. For example, modified RNA can refer to RNA with a 5′-cap; however, RNA may comprise further modifications. A 5′-cap can be modified to possess the ability to stabilize RNA when attached thereto. In certain embodiments, a further modification may be an extension or truncation of the naturally occurring poly(A) tail or an alteration of the 5′- or 3′-untranslated regions (UTR). In some embodiments, the RNA e.g.or mRNA vaccine is formulated in an effective amount to produce an antigen specific immune response in a subject. For example, the RNA vaccine formulation is administered to a subject in order to stimulate the humoral and/or cellular immune system of the subject against the Aβ and tau antigens, and thus may further comprise one or more adjuvant(s), diluents, carriers, and/or excipients, and is applied to the subject in any suitable route in order to elicit a protective and/or therapeutic immune reaction against the Aβ and tau antigens.

Basic texts disclosing general methods of molecular biology, all of which are incorporated by reference, include: Sambrook, J et al., Molecular Cloning: A Laboratory Manual, 2^{nd} Edition, Cold Spring Harbor Press, Cold Spring Harbor, N.Y., 1989; Ausubel, F M et al. Current Protocols in Molecular Biology, Vol. 2, Wiley-Interscience, New York, (current edition); Kriegler, Gene Transfer and Expression: A Laboratory Manual (1990); Glover, DM, ed, DNA Cloning: A Practical Approach, vol. I & II, IRL Press, 1985; Albers, B. et al., Molecular Biology of the Cell, 2^{nd} Ed., Garland Publishing, Inc., New York, N.Y. (1989); Watson, J D et al., Recombinant DNA, 2^{nd} Ed., Scientific American Books, New York, 1992; and Old, R W et al., Principles of Gene Manipulation: An Introduction to Genetic Engineering, 2^{nd} Ed., University of California Press, Berkeley, Calif. (1981).

Techniques for the manipulation ofnucleic acids, such as, e.g., generating mutations in sequences, sub-cloning, labeling probes, sequencing, hybridization and the like are well described in the scientific and patent literature. See, eg., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, radioimmunoassays (RIAs), enzyme-linked immunosorbent assays (ELISAs), immunofluorescence assays, Southern analysis, Northern analysis, dot-blot analysis, gel electrophoresis (e.g., SDS-PAGE), RT-PCR, quantitative PCR, other nucleic acid or target or signal amplification methods, radiolabeling, scintillation counting, and affinity chromatography.

### Pharmaceutical Compositions

Each of the peptides and immunogens described herein can be presented in a pharmaceutical composition that is administered with pharmaceutically acceptable adjuvants and pharmaceutically acceptable excipients. The adjuvant increases the titer of induced antibodies and/or the binding affinity of induced antibodies relative to the situation if the peptide were used alone. A variety of adjuvants can be used in combination with an immunogen of the disclosure to elicit an innnuae response. Some adjuvants augment the intrinsic response to an immunogen without causing conformational changes in the immunogen that affect the qualitative form of the response. An adjuvant may be a natural compound, a modified version of or derivative of a natural compound, or a synthetic compound.

Some adjuvants include aluminum salts, such as aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). As used herein, MPL refers to natural and synthetic versions of MPL. Examples of synthetic versions include PHAD^{®}, 3D-PHAD^{®} and 3D(6A)-PHAD^{®} (Avanti Polar Lipids (Croda), Alabaster, Alabama).

QS-21 is a triterpene glycoside or saponin isolated from the bark of the Quillaja Saponaria molina tree found in South America (see Kensil et al., in Vaccine Design: The Subunit and Adjuvant Approach (eds. Powell & Newman, Plenum Press, NY, 1995)) QS-21 products include Stimulon® (Antigenics, Inc., New York, NY; now Agenus, Inc. Lexington, MA) and QS-21 Vaccine Adjuvant (Desert King, San Diego, CA). QS-21 has been disclosed, characterized, and evaluated in US 5,057,540, and US 8,034,348 the disclosures of which are herein incorporated by reference. Additionally, QS-21 has been evaluated in numerous clinical trials in various dosages. See, NCT00960531 (clinicaltrials.gov/ct2/show/study/NCT00960531), Hüll et al., Curr Alzheimer Res. 2017 Jul; 14(7): 696-708 (evaluated 50 mcg of QS-21 in with various doses of vaccine ACC-001); Gilman S, et al., “Clinical effects of Abeta immunization (AN1792) in patients with AD in an interrupted trail “Neurology. 2005 May 10; 64(9):1553-62 Wald A, et al., Safety and immunogenicity of long HSV-2 peptides complexed with rhHsc70 in HSV-2 seropositive persons Vaccine 2011 3;29(47):8520-8529; and Cunningham et al., Efficacy of the Herpes Zoster Subunit Vaccine in Adults 70 Years of Age or Older. NEJM. 2016 Sep 15;3.75(11);1019.32.. QS-21 is used in FDA approved vaccines including SHINGRIX. SHINGRIX contains 50 mcg of QS-21 In certain embodiments, the amount of QS-21 is from about 10 µg to about 500 µg.

TQL1055 is an analogue of QS-21 (Adjuvance Technologies, Lincoln, NE). The semi-synthetic TQL1055 has been characterized in comparison to QS-21 as having high purity, increased stability. decreased local tolerability, decreased systemic tolerability. TQL1055 has been disclosed, characterized, and evaluated in US20180327436A1, WO2018191598A1, WO2018200656A1, and WO2019079160A1, the disclosures of which are herein incorporated by reference. US20180327436A1 teaches that 2.5 fold more TQ1055 was superior to 20 µg QS-21 but there was not an improvement over 50 µg TQ1055. However, unlike QS-21 there was no increase in either weight loss or hemolysis of RBC as the TQL1055 dose increased. WO2018200656A1 teaches that with an optimal amount of TQ1055, one can lower the amount of antigen and achieve superior titers. In certain embodiments, the amount of TQL1055 is from about 10 µg to about 500 µg.

Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Ribi adjuvants are oil-in-water emulsions. Ribi contains a metabolizable oil (squalene) emulsified with saline containing Tween 80. Ribi also contains refined mycobacterial products which act as immunostimulants and bacterial monophosphoryl lipid A. Other adjuvants can be CpG oliognucleotides (see WO 98/40100), cytokines (e.g., IL-1, IL-1 alpha and β peptides, IL-2, γ-INF, IL-10, GM-CSF), chemokines (e.g., MIP1-α and β, and RANTES), saponins, RNA, and/or TLR agonists (for example, TLR4 agonists such as MPL and synthetic MPL molecules), aminoalkyl glucosaminide phosphate and other TLR agonists. Adjuvants can be administered as a component of a therapeutic composition with an active agent or can be administered separately, before, concurrently with, or after administration of the therapeutic agent.

In various embodiments of the disclosure, the adjuvant is QS-21 (Stimulon™). In some compositions, the adjuvant is MPL. In certain embodiments, the amount of MPL is from about 10 µg to about 500 µg. In some compositions, the adjuvant is TQL1055, In certain embodiments, the amount of TQL1055 is from about 10 µg to about 500 µg. In some compositions, the adjuvant is QS21. In certain embodiments, the amount of QS21 is from about 10 µg to about 500 µg. In some compositions, the adjuvant is a combination of MPL and QS-21. In some compositions, the adjuvant is a combination of MPL and TQL10535. In some compositions, the adjuvant can be in a liposomal formulation.

In addition, some embodiments of the disclosure can comprise a multiple antigen presenting system (MAP). Multiple antigen-presenting peptide vaccine systems have been developed to avoid the adverse effects associated with conventional vaccines (ie., live-attenuated, killed or inactivated pathogens), carrier proteins and cytotoxic adjuvants. Two main approaches have been used to develop multiple antigen presenting peptide vaccine systems: (1) the addition of functional components, e.g., T-cell epitopes, cell-penetrating peptides, and lipophilic moieties; and (2) synthetic approaches using size-defined nanomaterials, e.g., self-assembling peptides, non-peptidic dendrimers, and gold nanoparticles, as antigen-displaying platforms. Use of a multiple antigenic peptide (MAP) system can improve the sometimes poor immunogenicity of subunit peptide vaccines. In a MAP system, multiple copies of antigenic peptides are simultaneously bound to the a- and ε-amino groups of a non-immunogenic Lys-based dendritic scaffold, helping to confer stability from degradation, thus enhancing molecular recognition by immune cells, and induction of stronger immune responses compared with small antigenic peptides alone. In some compositions, the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the peptides of the disclosure can be formulated in aqueous solutions, preferably in physiologically compatible buffers such as Hank’s solution, Ringer’s solution, or physiological saline or acetate buffer (to reduce discomfort at the site of injection). The solution can contain formulatory agents such as suspending, stabilizing and/or dispersing agents. Alternatively, peptide compositions can be in lyophilized form for constitution with a suitable vehicle, e.g., sterile pyrogen-free water, before use.

Peptides (and optionally a carrier fused to the peptide(s)) can also be administered in the form of a nucleic acid encoding the peptide(s) and expressed in situ in a subject. A nucleic acid segment encoding an immunogen is typically linked to regulatory elements, such as a promoter and enhancer that allow expression of the DNA segment in the intended target cells of a subject. For expression in blood cells, as is desirable for induction of an immune response, promoter and enhancer elements from, for example, light or heavy chain immunoglobulin genes or the CMV major intermediate early promoter and enhancer are suitable to direct expression. The linked regulatory elements and coding sequences are often cloned into a vector.

DNA and RNA can be delivered in naked form (i.e., without colloidal or encapsulating materials). Alternatively, a number of viral vector systems can be used including retroviral systems (see, e.g., Boris-Lawrie and Teumin, Cur. Opin, Genet Develop, 3, 102-109 (1993)); adenoviral vectors (see, e.g., Bett et al, J. Virol. 67(10), 591 1 (1993)); adeno-associated virus vectors (see, e.g., Zhou et αl., J. Exp. Med. 179, 1867 (1994)), viral vectors from the pox family including vaccinia virus and the avian pox viruses, viral vectors from the alpha virus genus such as those derived from Sindbis and Semliki Forest Viruses (see, e.g., Dubensky et al., J. Virol. 70, 508-519 (1996)), Venezuelan equine encephalitis virus (see US 5,643,576) and rhabdoviruses, such as vesicular stomatitis virus (see WO 96/34625)and papillomaviruses (Ohe et al., Human Gene Therapy 6, 325-333 (1995); Woo et al, WO 94/12629 and Xiao & Brandsma, Nucleic Acids. Res. 24, 2620-2622 (1996)).

DNA and RNA encoding an immunogen, or a vector containing the same, can be packaged into liposomes, nanoparticles or lipoproteins complexes. Other suitable polymers, include, for example, protamine liposomes, polysaccharide particles, cationic nanoemulsion, cationic polymer, cationic polymer liposome, cationic lipid nanoparticles, cationic lipid, cholesterol nanoparticles, cationic lipid-cholesterol, PEG nanoparticle, or dendrimer nanoparticles. Additional suitable lipids and related analogs are described by US 5,208,036, US 5,264,618, US 5,279,833, and US 5,283,185, each of which is incorporated by reference in their entirety. Vectors and DNA encoding an immunogen can also be adsorbed to or associated with particulate carriers, examples of which include polymethyl methacrylate polymers and polylactides and poly(lactide-co-glycolides), (see, e.g., McGee et al., J. Micro Encap. 1997; 14(2):197-210).

Pharmaceutically acceptable carrier compositions can also include additives, including but not limited to water, pharmaceutically acceptable organic solvents, collagen, polyvinyl alcohol, polyvinylpyrrolidone, carboxyvinyl polymers, carboxymethylcellulose sodium, sodium polyacrylate, sodium alginate, water-soluble dextran, carboxymethyl starch sodium, pectin, methylcellulose, ethylcellulose, xanthan gum, gum arabic, casein, agar, polyethylene glycol, diglycerine, glycerine, propylene glycol, petrolatum, paraffin, stearyl alcohol, stearic acid, human serum albumin, mannitol, sorbitol, lactose, and surfactants acceptable as pharmaceutical additives.

### Subjects Amenable to Treatment

The presence of Aβ plaques has been found in several diseases including Alzheimer’s disease, Down’s syndrome, mild cognitive impairment, cerebral amyloid angiopathy, postencephalitic parkinsonism, posttraumatic dementia or dementia pugilistica, Pick’s disease, type C Niemann-Pick disease, supranuclear palsy, frontotemporal dementia, frontotemporal lobar degeneration, argyrophilic grain disease, amyotrophic lateral sclerosis/parkinsonism dementia complex of Guam, corticobasal degeneration (CBD), dementia with Lewy bodies, Lewy body variant of Alzheimer’s disease (LBVAD), chronic traumatic encephalopathy (CTE), Parkinson’s disease, progressive supranuclear palsy (PSP), dry age-related macular degeneration (AMD), and inclusion-body myositis.

The compositions and methods of the disclosure can be used in treatment or prophylaxis of any of these diseases. Because of the widespread association between neurological diseases and Aβ, the compositions and methods of the disclosure can be used in treatment or prophylaxis of any subject showing elevated levels of Aβ (e.g., in the CSF) compared with a mean value in individuals without neurological disease. The compositions and methods of the disclosure can also be used in treatment or prophylaxis of neurological disease in individuals having a mutation in Aβ associated with neurological disease. The methods are particularly suitable for treatment or prophylaxis of Alzheimer’s disease.

Subjects amenable to treatment include individuals at risk of disease but not showing symptoms, as well as patients presently showing symptoms, inchiding treatment naive subjects that have not been previous treated for disease. Subjects at risk of disease include those in an aging population, asymptomatic subjects with Aβ pathologies and having a known genetic risk of disease. Such individuals include those having relatives who have experienced this disease, and those whose risk is determined by analysis of genetic or biochemical markers. Genetic markers of risk include mutations in Aβ, as well as mutations in other genes associated with neurological disease. For example, the ApoE4 allele in heterozygous and even more so in homozygous form is associated with risk of Alzheimer’s disease (AD). Other markers of risk of Alzheimer’s disease include mutations in the APP gene, particularly mutations at position 717 and positions 670 and 671 referred to as the Hardy and Swedish mutations respectively, mutations in the presenilin genes, PS1 and PS2, a family history of AD, hypercholesterolemia or atherosclerosis. Individuals presently suffering from Alzheimer’s disease can be recognized by PET imaging, from characteristic dementia, as well as the presence of risk factors described above. In addition, a number of diagnostic tests are available for identifying individuals who have AD. These include measurement of CSF or blood Aβ42 levels. Decreased Aβ42 levels can signify the presence of AD, as well as increased Aβ40 and a reduced Aβ42/Aβ40 ratio. Some mutations associated with Parkinson’s disease, for example, Ala30Pro or Ala53Thr, or mutations in other genes associated with Parkinson’s disease such as leucine-rich repeat kinase (LRRK2 or PARKS). Subjects can also be diagnosed with any of the neurological diseases mentioned above by the criteria of the DSM IV TR.

In asymptomatic subjects, treatment can begin at any age (e.g., 10, 20, 30, or more). Usually, however, it is not necessary to begin treatment until a subject reaches 20, 30, 40, 50, 60, 70, 80 or 90 years of age. Treatment typically entails multiple dosages over a period of time. Treatment can be monitored by assaying antibody levels over time. If the response falls, a booster dosage is indicated. In the case of potential Down’s syndrome patients, treatment can begin antenatally by administering therapeutic agent to the mother or shortly after birth.

### Methods of Treatments and Uses

The disclosure provides methods of inhibiting or reducing aggregation of Aβ in a subject having or at risk of developing Alzheimer’s disease. The methods include administering to the subject the compositions as disclosed herein. A therapeutically effective amount is a dosage that, when given for an effective period of time, achieves the desired immunological or clinical effect. Dosage regimens may be adjusted to provide the optimum therapeutic response. For example, several divided doses may be administered at set intervals (e.g., weekly, monthly) or the dose may be proportionally reduced as indicated by the exigencies of the therapeutic situation.

The compositions described herein (pharmaceutical compositions) can be used in methods for treating or effecting prophylaxis and/or prevention of Alzheimer’s disease. In certain embodiments, the compositions as disclosed herein provide compositions for reducing Aβ in a subject and/or in the tissue of the subject. In another embodiment, the compositions as disclosed herein provide compositions for reducing Aβ in the brain of a subject. In some embodiments, the Aβ reduced by the compositions is the pathological form(s) of the Aβ (e.g. extracellular plaque deposits of the β-amyloid peptide (Aβ); neuritic amyloid plaques). In yet other embodiment, pathological indicators of neurodegenerative disease and/or β-amyloidopathies are decreased by the immunotherapy compositions.

In prophylactic applications, the compositions described herein can be administered to a subject susceptible to, or otherwise at risk of a disease (e.g., Alzheimer’s disease) in a regimen (dose, frequency and route of administration) effective to reduce the risk lessen the severity, or delay the onset of at least one sign or symptom of the disease. In particular, the regimen is effective to inhibit or delay Aβ plaque formation, and/or inhibit or delay its toxic effects and/or inhibit/or delay development of behavioral deficits. In therapeutic applications, the compositions described herein are administered to a subject suspected of, or a patient already suffering from a disease (e.g., Alzheimer’s disease) in a regimen (dose, frequency and route of administration) effective to ameliorate or at least inhibit further deterioration of at least one sign or symptom of the disease. In particular, the regimen is preferably effective to reduce or at least inhibit further increase of levels of Aβ plaques associated toxicities and/or behavioral deficits.

A regimen is considered therapeutically or prophylactically effective if an individual treated achieves an outcome more favorable than the mean outcome in a control population of comparable subjects not treated by methods of the invention, or if a more favorable outcome is demonstrated in treated subjects versus control subjects in a controlled clinical trial (e.g., a phase II, phase II/III or phase III trial) at the p < 0.05 or 0.01 or even 0.001 level.

Effective doses of vary depending on many different factors, such as means of administration, target site, physiological state of the patient, whether the patient is an ApoE carrier, whether the patient is human or an animal, other medications administered, and whether treatment is prophylactic or therapeutic.

In some embodiments, the effective amount is a total dose of 25 µg to 1000 µg, or 50 µg to 1000 µg. In some embodiments, the effective amount is a total dose of 100 µg. In some embodiments, the effective amount is a dose of 25 µg administered to the subject a total of two times. In some embodiments, the effective amount is a dose of 100 µg administered to the subject a total of two times. In some embodiments, the effective amount is a dose of 400 µg administered to the subject a total of two times. In some embodiments, the effective amount is a dose of 500 µg administered to the subject a total of two times. In some embodiments, a RNA (e.g., mRNA) vaccine is administered to a subject by intradermal, intramuscular injection, or by intranasal administration.

In some embodiments, the amount of an agent for active immunotherapy varies from 1 to 1,000 micrograms (µg), or from 0.1-500 µg, or from 10 to 500 µg, or from 50 to 250 µg per patient and can be from 1-100 or 1-10 µg per injection for human administration. The timing of injections can vary significantly from once a day, to once a week, to once a month, to once a year, to once a decade. A typical regimen consists of an immunization followed by booster injections at time intervals, such as 6 week intervals or two months. Another regimen consists of an immunization followed by one or more booster injections 1, 2, 3, 4, 5, 6, or 12 months later. Another regimen entails an injection every two months for life. Alternatively, booster injections can be on an irregular basis as indicated by monitoring of immune response. The frequency of administration may be once or more as long as the side effects are within a clinically acceptable range.

In some embodiments, the compositions or methods as disclosed herein comprise administering to a subject a nucleic acid vaccine comprising one or more DNA or RNA polynucleotides having an open reading frame encoding a first peptide and a second peptide wherein a dosage of between 10 µg/kg and 400 µg /kg of the nucleic acid vaccine is administered to the subject. In some embodiments the dosage of the RNA polynucleotide is 1-5 µg, 5-10 µg, 10-15 µg, 15-20 µg, 10-25 µg, 20-25 µg, 20-50 µg, 30-50 µg, 40-50 µg, 40-60 µg, 60-80 µg, 60-100 µg, 50-100 µg, 80-120 µg, 40-120 µg, 40-150 µg, 50-150 µg, 50-200 µg, 80-200 µg, 100-200 µg, 120-250 µg, 150-250 µg, 180-280 µg, 200-300 µg, 50-300 µg, 80-300 µg, 100-300 µg, 40-300 µg, 50-350 µg, 100-350 µg, 200-350 µg, 300-350 µg, 320-400 µg, 40-380 µg, 40-100 µg, 100-400 µg, 200-400 µg, or 300-400 µg per dose. In some embodiments, the nucleic acid is administered to the subject by intradermal or intramuscular injection. In some embodiments, the nucleic acid is administered to the subject on day zero. In some embodiments, a second dose of the nucleic acid is administered to the subject on day seven, or fourteen, or twenty one.

The compositions described herein are preferably administered via a peripheral route (i.e., one in which the administered composition results in a robust immune response and/or the induced antibody population crosses the blood brain barrier to reach an intended site in the brain, spinal cord, or eye. For peripheral diseases, the induced antibodies leave the vasculature to reach the intended peripheral organs. Routes of administration include oral, subcutaneous, intranasal, intradermal, or intramuscular. Some routes for active immunization are subcutaneous and intramuscular. Intramuscular administration and subcutaneous administration can be made at a single site or multiple sites. Intramuscular injection is most typically performed in the arm or leg muscles. In some methods, agents are injected directly into a particular tissue where deposits have accumulated.

The number of dosages administered can be adjusted to result in a more robust immune response (for example, higher titers).

An effective amount of a DNA or RNA encoded immunogen can be between about 1 nanogram and about 1 gram per kilogram of body weight of the recipient, or about between about 0.1 µg/kg and about 10 mg/kg, or about between about 1 µg/kg and about 1 mg/kg. Dosage forms suitable for internal administration preferably contain (for the latter dose range) from about 0.1 µg to 100 µg of active ingredient per unit. The active ingredient may vary from 0.5 to 95% by weight based on the total weight of the composition. Alternatively, an effective dose of dendritic cells loaded with the antigen is between about 10⁴ and 10⁸ cells. Those skilled in the art of immunotherapy will be able to adjust these doses without undue experimentation.

The nucleic acid compositions may be administered in a convenient manner, e.g., injection by a convenient and effective route. Routes can include, but are not limited to, intradermal “gene gun” delivery or intramuscular injection. The modified dendritic cells are administered by subcutaneous, intravenous or intramuscular routes. Other possible routes include oral administration, intrathecal, inhalation, transdermal application, or rectal administration.

Depending on the route of administration, the composition may be coated in a material to protect the compound from the action of enzymes, acids and other natural conditions which may inactivate the compound. Thus, it may be necessary to coat the composition with, or co-administer the composition with, a material to prevent its inactivation. For example, an enzyme inhibitors of nucleases or proteases (e.g., pancreatic trypsin inhibitor, diisopropylfluorophosphate and trasylol) or in an appropriate carrier such as liposomes (including water-in-oil-in-water emulsions as well as conventional liposomes (Strejan et al., J. Neuroimmunol 7:27, 1984).

The immunotherapeutic compositions disclosed herein may also be used in combination with other treatments for diseases associated with the accumulation of Aβ, for example, anti-Aβ antibodies such as antibodies that specifically bind to any of the Aβ epitopes disclosed herein, aducanumab or any of the antibodies disclosed in, for example, U.S. Pat. Publication 2010/202968 and U.S. Pat. No. 8,906,367, ABBV-8E12, gosuranemab, zagotenemab, RG-6100, BIIB076 or any of the antibodies disclosed in WO2014/165271, US10,501,531, WO-201-71191560, US2019/0330314, WO-20171191561, LUS2019/0330316, WO201-7/191559, and WO2018/204546. In some combination therapy methods, the patient receives passive immunotherapy prior to the active immunotherapy methods disclosed herein. In other methods, the patient receives passive and active immunotherapy during the same period of treatment. Alternatively, patients may receive active immunotherapy prior to passive immunotherapy. Combinations may also include small molecule therapies and non-immunogenic therapies such as RAZADYNE® (galantamine), EXELON® (rivastigmine), and ARICEPT® (donepezil) and other compositions that improve the function of nerve cells in the brain.

The compositions of the disclosure may be used in the manufacture of medicaments for the treatment regimens described herein.

### Treatment Regimens

Desired outcomes of the methods of treatment as disclosed herein vary according to the disease and patient profile and are determinable to those skilled in the art. Desired outcomes include an improvement in the patient’s health status. Generally, desired outcomes include measurable indices such as reduction or clearance of pathologic amyloid fibrils, decreased or inhibited amyloid aggregation and/or deposition of amyloid fibrils, and increased immune response to pathologic and/or aggregated amyloid fibrils. Desired outcomes also include amelioration of amyloid disease-specific symptoms. As used herein, relative terms such as “improve,” “increase,” or “reduce” indicate values relative to a control, such as a measurement in the same individual prior to initiation of treatment described herein, or a measurement in a control individual or group. A control individual is an individual afflicted with the same amyloid disease as the individual being treated, who is about the same age as the individual being treated (to ensure that the stages of the disease in the treated individual and the control individual are comparable), but who has not received treatment using the disclosed formulations. Alternatively, a control individual is a healthy individual, who is about the same age as the individual being treated. Changes or improvements in response to therapy are generally statistically significant and described by a p-value less than or equal to 0.1, less than 0.05, less than 0.01, less than 0.005, or less than 0.001 may be regarded as significant.

Effective doses of the compositions as disclosed herein, for the treatment of a subject vary depending upon many different factors, including means of administration, target site, physiological state of the patient, whether the patient is human or an animal, other medications administered, if any, and whether treatment is prophylactic or therapeutic. Treatment dosages can be titrated to optimize safety and efficacy. The amount of immunogen can also depend on whether adjuvant is also administered, with higher dosages being required in the absence of adjuvant. The amount of an immunogen for administration sometimes varies from 1-500 µg per patient and more usually from 5-500 µg per injection for human administration. Occasionally, a higher dose of 1-2 mg per dosage is used. Typically, about 10, 20, 50 or 100 µg is used for each human dosage. The timing of dosages can vary significantly from once a day, to once a year, to once a decade. On any given day that a dosage of immunogen is given, the dosage is greater than 1 µg/patient and usually greater than 10 µg/patient if adjuvant is also administered, and greater than 10 µg/patient and usually greater than 100 µg/patient in the absence of adjuvant. A typical regimen consists of an immunization followed by booster dosage(s) at 6-week intervals. Another regimen consists of an immunization followed by booster dosage(s) 1, 2, 3, 4, 5, 6, or 12 months later. Another regimen entails dosage(s) every two months for life. Alternatively, booster dosage(s) can be on an irregular basis as indicated by monitoring of immune response.

When administered in combination with a second treatment for Alzheimer’s disease, such as, Razadyne® (galantamine), Exelon® (rivastigmine), and Aricept® (donepezil), the second treatment can be administered according the product label or as necessary in view of the treatment with the compositions of the disclosure.

### Kits

The disclosure further provides kits (e.g., containers) comprising the compositions disclosed herein and related materials, such as instructions for use (e.g., package insert). The instructions for use may contain, for example, instructions for administration of the compositions and optionally one or more additional agents. The containers of peptide and/or nucleic acid compositions may be unit doses, bulk packages (e.g., multi-dose packages), or sub-unit doses.

Package insert refers to instructions customarily included in commercial packages of therapeutic products that contain information about the indications, usage, dosage, administration, contraindications and/or warnings concerning the use of such therapeutic products. Kits can also include a second container comprising a pharmaceutically acceptable buffer, such as bacteriostatic water for injection (BWEI), phosphate-buffered saline, Ringer’s solution and dextrose solution. It can also include other materials desirable from a commercial and user standpoint, including other buffers, diluents, filters, needles, and syringes.

The following are provided for exemplification purposes only and are not intended to limit the scope of the invention described in broad term above. All references cited in this disclosure are incorporated herein by reference.

### Uses

Each of the peptides, polypeptides, immunogens, and pharmaceutical compositions described herein may be for use in treating one or more of the diseases as described herein. In addition, each of the peptides, polypeptides, immunogens, and pharmaceutical compositions described herein may be for use in methods for treating one or more of the diseases as described herein. Each of the peptides, polypeptides, immunogens, and pharmaceutical compositions described herein may be used in a method for manufacturing a medicament for treating or use in treating one or more of the diseases as described herein.

All U.S. and international patent applications identified herein are incorporated by reference in their entirety.

### EXAMPLES

### Example 1: Animal Immunizations

Female Swiss Webster mice were injected subcataneously at two sites with 100 µl of test article on day 0, 14, 42 and 70. Test article was prepared by combining 25 µg of test immunogen and 25 µg of QS21 adjuvant in 200 µl phosphate buffered saline (PBS). Mice were bled on day 21, 49 and 77 by nicking tails and collecting 50 µl of blood, followed by processing to serum. The peptides tested included AEFRHDSGC (SEQ ID NO:38) and DAEFRHDC (SEQ ID NO:39). Immunogens contained one Aβ peptide, a C-terminal linker and a C-terminal cysteine and were coupled through the C-terminal cysteine to CRM-197 with a maleimide linkage.

Guinea pigs were injected intramuscularly with 50 µg of a test immunogen, 25 µg QS21 in 200 µl of Addavax on day 0, 21, 49 and 77. Bleeds were done 7 days post immunization. The peptides tested included DAEFRHDC (SEQ ID NO:39) and QKLVFFAEC (SEQ ID NO:40). Immunogens again contained one Aβ peptide, a C-terminal linker. and a C-terminal cysteine and were coupled through the C-terminal cysteineto CRM-197 with a maleimide linkage.

Female Guinea Pigs were at least 5 weeks old at the start of the study having an approximate body weight of 350-500 g. Appropriate animal housing and research procedures for animal husbandry and care were conducted in an accredited facility in accordance with the guidelines of the U.S. Department of Agriculture’s (USDA) and the Assessment and Accreditation of Laboratory Animal Care (AAALAC) International

The immunogen concentration was 0.5 mg/ml. Prior to each administration of the test immunogen, approximately a 3 cm² area on each hind limb was shaved and wiped with ethanol for visualization of the injection site. Each animal received a test immunogen dose of 200 microliters (0.25 micrograms/microliter) divided into two separate sites each of 100 microliter per injection (i.e., animals received 50 µg of immunogen in 100 µl PBS + 25 µg of QS-21 in 100 µl Addavax). A 25G-27G needle was inserted intramuscularly into the hind limb, approximately 0.25 - 0.5 cm deep, and injected at 100 microliters per site. Injection sites were rotated each administration between four separate sites per hind limb and separated by at least 2 cm.

### Example 2: Measurement of Antibody Titers

Whole blood samples were collected into clot activator tubes via jugular vein at 250-350 microliters per collection at weeks 1, 4, 8 and 12 for Guinea pigs and 50 microliters per collection at weeks 1, 3, 7 and 11 by nicking tails for mice. The maximum volume of whole blood was collected into clot activator tubes via cardiac puncture at termination on the final collection week. All blood samples were allowed to clot at room temperature for greater than 30 minutes, centrifuged ambient (approximately 20-25° C.) at 3,000 RPM for 10-15 minutes, and serum supernatant was transferred individually into clean cryovials. Serum supernatant was stored frozen at -80° C. (± 12° C.).

### Titers on Aβ Guinea Pig

Aβ 1-15 and Aβ 1-28 were both used at different parts of the study. Both of these will not form aggregates. 2 µg/ml A-beta monomers were coated at coated on to the plate 100 µl per well in PBS and incubated overnight at room temperature. Plates were blocked for 1 hour with 1% BSA in PBS. Plates were aspirated and to row A 200 µl of 0.1% BSA in PBS Tween was added. In column 1 negative Guinea pig serum was added at 1/100 dilution while the rest of the row contained 1/100 test serums. Rows were serially diluted by 50% per step down the plate giving dilution range of 1/100 to 1/12800, Wells were incubated for 2 hours at room temperature then were washed. A 1/5000 dilution of anti-Guinea pig IgG HRP in 0.1% BSA in PBS Tween was prepared and then 100 µl added to the washed well. Samples were incubated for 1 hour and then washed. OPD substrate was prepared using Thermo-Fisher OPD tablets at 1 tablet per 10 mls. Thermofisher substrate buffer was added at ⅒ and each well had 100 µl added and was incubated for 15 minutes. 50 µl of 2N H₂SO₄ was added to stop the reaction and plates were read at 490 nm on a Molecular Devices Spectromax. Titer defined as the dilution giving 50% maximum OD and was extrapolated if it fell between dilutions.

### Titers on Aβ Mouse

2 µg/ml recombinant Aβ was coated on to the plate 100 µl per well in PBS and incubated overnight at room temperature. Plates were blocked for 1 hour with 1% BSA in PBS. Plates were aspirated and to row A 200 µl of 0.1% BSA in PBS Tween was added. In column 1, negative mouse serum was added at 1/100 while the rest of the row contained 1/100 test sera. Rows were serially diluted by 50% per step down the plate giving dilution of 1/100 to 1/12800. Wells were incubated 2 hours at room temperature then were washed, A 1/5000 dilution of anti-mouse IgG HRP in 0.1% BSA in PBS Tween was prepared and then 100 µl added to the washed well. The reaction mixture was incubated for 1 hour and was washed. OPD substrate was prepared using ThermoFisher OPD tablets at 1 tablet per 10 mls. ThermoFisher substrate buffer was added at ⅒ dilution and each well received 100 µl and was incubated for 15 minutes. 50 µl of 2N H₂SO₄ was added to stop the reaction and plates were read at 490 nm on a Molecular Devices Spectromax. Titer was defined as the dilution giving 50% maximum OD measurement and was extrapolated if it fell between dilutions.

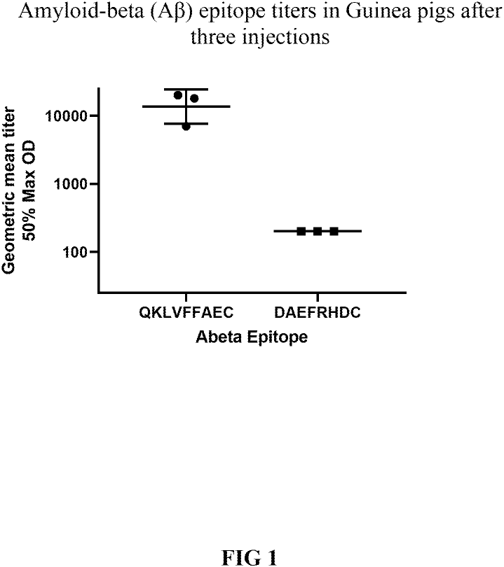

Antibody titers observed in Guinea pigs immunized as described above are shown in Table 1. Immunizations were conducted with QS21 in Addavax. The titers reported are for the bleed after the third injection. These results are represented in FIG. 1.

TABLE 1

Antibody titers in Guinea pigs (GP) immunized with Aβ epitopes.

| Aβ Epitope in immunogen | SEQ ID | GP 1 Titer | GP 2 Titer | GP 3 Titer |
|---|---|---|---|---|
| QKLVFFAEC Abeta 15-22 | 40 | 7000 | 20000 | 18000 |
| DAERFHDC Abeta 1-7 | 39 | 200 | 200 | 200 |

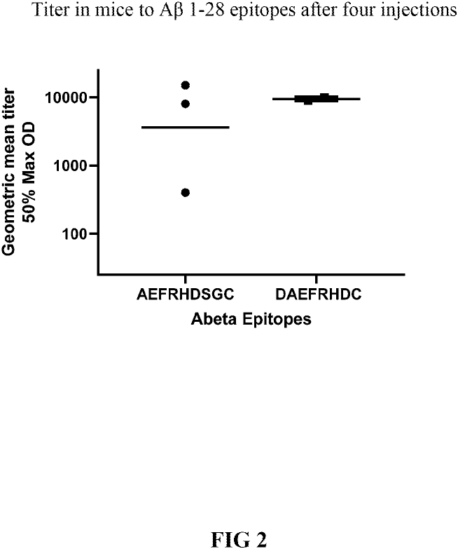

Antibody titers observed in mice immunized as described above are shown in Table 2. Immunizations were conducted with QS21. The titers reported are for the bleed after the third injection. These results are represented in FIG. 2.

TABLE 2

Antibody titers in mice immunized with Aβ epitopes.

| Aβ Epitope in immunogen | SEQ ID | Mouse 1 Titer | Mouse 2 Titer | Mouse 3 Titer | Mouse 4 Titer |
|---|---|---|---|---|---|
| AEFRHDSGC Abeta 2-8 | 38 | 8000 | 15000 | 400 | |
| DAEFRHDC Abeta 1-7 | 39 | 9000 | | | 10000 |

### Example 3: Staining of Alzheimer’s Brain Tissue With Sera From Guinea Pigs Immunized With a Vaccine as Disclosed Herein.

Autopsy blocks of fresh frozen human brain tissue (~ 0.5 g) are embedded in optimal cutting temperature compound (OCT compound) and cut using a cryostat to generate 10 µm sections. The sections are placed into a solution of glucose oxidase and beta D-glucose, in the presence of sodium aside, to block endogenous peroxidase. Once tissue sections are prepared, the staining with the specified Guinea pig sera from Guinea pigs immunized with a vaccine as disclosed herein is carried out at two dilutions (1:300 and 1:1500), using a rabbit anti-guinea pig secondary antibody and a DAKO DAB Detection Kit as per the manufacturer’s instructions. The staining is processed using an automated Leica Bond Stainer. The results indicate whether sera from Guinea pigs immunized with a vaccine as disclosed herein comprise antibodies specific to Aβ in human brain tissue of Alzheimer’s patients.

Although various specific embodiments of the present invention have been described herein, it is to be understood that the invention is not limited to those precise embodiments and that various changes or modifications can be affected therein by one skilled in the art without departing from the scope and spirit of the invention.

In each of the embodiments of the peptide described herein, the peptide may comprise, consist, or consist essentially of the recited sequences. Thus, incorporated in this disclosure (see Table 13) are the following sequences that can be part of the compositions comprising, consisting of or consisting essentially of an amyloid-beta (Aβ) peptide as disclosed herein.

TABLE 3

SEQ ID NO:01 - Aβ1 -42 DAEFRHDSGYEVHHQKLVFFAEDVGSNKGAIIGLMVGGVVIA

| | | | |
|---|---|---|---|
| DABFRHDSGY | (SEQ ID NO:02) | DSGY | (SEQ ID NO:35) |
| DAEFRHDSG | (SEQ ID NO:03) | DSG | (SEQ ID NO:36) |
| DAEFRHDS | (SEQ ID NO:04) | SGY | (SEQ ID NO:37) |
| DAEFRHD | (SEQ ID NO:05) | AEFRHDSGC | (SEQ ID NO:38) |
| DAEFRH | (SEQ ID NO:06) | DAEFRHDC | (SEQ ID NO:39) |
| DAEFR | (SEQ ID NO:07) | | |
| DAEF | (SEQ ID NO:08) | QKLVFFAEC | (SEQ ID NO:40) |
| DAE | (SEQ ID NO:09) | VHHQKLVFFA | (SEQ ID NO:41) |
| AEFRHDSGY | (SEQ ID NO:10) | VHHQKLVFF | (SEQ ID NO:42) |
| AEFRHDSG | (SEQ ID NO:11) | VHHQKLVF | (SEQ ID NO:43) |
| AEFRHDS | (SEQ ID NO:12) | VHHQKLV | (SEQ ID NO:44) |
| AEFRHD | (SEQ ID NO:13) | VHHQKL | (SEQ ID NO:45) |
| AEFRH | (SEQ ID NO:14) | HHQKLVFFAE | (SEQ ID NO:46) |
| AEFR | (SEQ ID NO:15) | HHQKLVFFA | (SEQ ID NO:47) |
| AEF | (SEQ ID NO:16) | HHQKLVFF | (SEQ ID NO:48) |
| EFRHDSGY | (SEQ ID NO:17) | HHQKLVF | (SEQ ID NO:49) |
| EFRHDSG | (SEQ ID NO:18) | HHQKLV | (SEQ ID NO:50) |
| EFRHDS | (SEQ ID NO:19) | HHQKL | (SEQ ID NO:51) |
| EFRHD | (SEQ ID NO:20) | HQKLVFFAED | (SEQ ID NO:52) |
| EFRH | (SEQ ID NO:21) | HQKLVFFAE | (SEQ ID NO:53) |
| EFR | (SEQ ID NO:22) | HQKLVFFA | (SEQ ID NO:54) |
| FRHDSGY | (SEQ ID NO:23) | HQKLVFF | (SEQ ID NO:55) |
| FRRDSG | (SEQ ID NO:24) | HQKLVF | (SEQ ID NO:56) |
| FRHDS | (SEQ ID NO:25) | HQKLV | (SEQ ID NO:57) |
| FRHD | (SEQ ID NO:26) | HQKL | (SEQ ID NO:58) |
| FRH | (SEQ ID NO:27) | QKLVFFAEDV | (SEQ ID NO:59) |
| RHDSGY | (SEQ ID NO:28) | QKLVFFAED | (SEQ ID NO:60) |
| RHDSG | (SEQ ID NO:29) | QKLVFFAE | (SEQ ID NO:61) |
| RHDS | (SEQ ID NO:30) | QKLVFFA | (SEQ ID NO:62) |
| RHD | (SEQ ID NO:31) | QKLVFF | (SEQ ID NO:63) |
| HDSGY | (SEQ ID NO:32) | QKLVF | (SEQ ID NO:64) |
| HDSG | (SEQ ID NO:33) | QKLV | (SEQ ID NO:65) |
| HDS | (SEQ ID NO:34) | QKL | (SEQ ID NO:66) |
| KLVFFAEDVG | (SEQ ID NO:67) | VFFAE | (SEQ ID NO:85) |
| KLVFFAEDV | (SEQ ID NO:68) | VFFA | (SEQ ID NO:86) |
| KLVFFAED | (SEQ ID NO:69) | VFF | (SEQ ID NO:87) |
| KLVFFAE | (SEQ ID NO:70) | FFAEDVG | (SEQ ID NO:88) |
| KLVFFA | (SEQ ID NO:71) | FFAEDV | (SEQ ID NO:89) |
| KLVFF | (SEQ ID NO:72) | FFAED | (SEQ ID NO:90) |
| KLVF | (SEQ ID NO:73) | FFAE | (SEQ ID NO:91) |
| KLV | (SEQ ID NO:74) | FFA | (SEQ ID NO:92) |
| LVFFAEDVG | (SEQ ID NO:75) | FAEDVG | (SEQ ID NO:93) |
| LVFFAEDV | (SEQ ID NO:76) | FAEDV | (SEQ ID NO:94) |
| LVFFAED | (SEQ ID NO:77) | FAED | (SEQ ID NO:95) |
| LVFFAE | (SEQ ID NO:78) | FAE | (SEQ ID NO:96) |
| LVFFA | (SEQ ID NO:79) | Arg-Val-Arg-Arg | (RVRR; SEQ ID NO:97) |
| LVFF | (SEQ ID NO:80) | Gly-Ala-Gly-Ala | (GAGA; SEQ ID NO:98) |
| LVF | (SEQ ID NO:81) | Ala-Gly-Ala-Gly | (AGAG; SEQ ID NO:99) |
| VFFAEDVG | (SEQ ID NO:82) | Lys-Gly-Lys-Gly | (KGKG; SEQ ID NO:100) |
| VFFAEDV | (SEQ ID NO:83) | DAEFRHDRR | (SEQ ID NO: 101). |
| VFFAED | (SEQ ID NO:84) | | |

What is claimed is:

1. A peptide comprising 3-10 amino acids from residues 1-10 of SEQ ID NO:01 or from residues 12-25 of SEQ ID NO:01.

2. The peptide of claim 1, wherein the peptide comprises an amino acid sequence selected from the group consisting of any one of SEQ ID NO:02 to SEQ ID NO:37 or SEQ ID NO:41 to SEQ ID NO:96.

3. The peptide of claim 1, wherein the peptide is from residues 1-7 of SEQ ID NO:01.

4. The peptide of claim 1, wherein the peptide is from residues 12-24 or from residues 12-23 or from residues 12-22 or from residues 13-25 or from residues 13-24 or from residues 13-23 or from residues 13-22 or from residues 14-25 or from residues 14-24 or from residues 14-23 or from residues 14-22 or from residues 15-25 or from residues 15-24 or from residues 15-23 or from residues 15-22 of SEQ ID NO:01.

5. The peptide of claim 1, wherein the peptide comprises an amino acid sequence selected from the group consisting of any one of SEQ ID NO:05 to SEQ ID NO:09, SEQ ID NO:13 to SEQ ID NO: 16, SEQ ID NO:20 to SEQ ID NO:22, SEQ ID NO:26, SEQ ID NO:27, or SEQ ID NO:31.

6. The peptide of claim 1, wherein the peptide is from residues 2-8 of SEQ ID NO:01.

7. The peptide of claim 1, wherein the peptide comprises an amino acid sequence selected from the group consisting of one of SEQ ID NO: 12 to SEQ ID NO: 16, SEQ ID NO: 19 to SEQ ID NO:22, SEQ ID NO:25 to SEQ ID NO:27, SEQ ID NO:30, SEQ ID NO:31 or SEQ ID NO:34.

8. The peptide of claim 1, wherein the peptide comprises an amino acid sequence selected from the group consisting of SEQ ID NOS 2-96.

9. The peptide of claim 8, further comprising a -RR at a C-terminal end.

10. The peptide of claim 9, comprising an amino acid sequence of DAEFRHDRR (SEQ ID NO:101).

11. The peptide of any one of claims 1-10, further comprising a C-terminal cysteine.

12. The peptide of claim 1, comprising an amino acid sequence of AEFRHDSGC (SEQ ID NO:38).

13. The peptide of claim 1, comprising an amino acid sequence of DAEFRHDC (SEQ ID NO:39).

14. The peptide of claim 1, comprising an amino acid sequence of QKLVFFAEC (SEQ ID NO:40).

15. The peptide of claim 1, comprising an amino acid sequence of DAEFRHD (SEQ ID NO:05).

16. The peptide of claim 1, comprising an amino acid sequence of EFRHDSG (SEQ ID NO:18).

17. The peptide of claim 1, comprising an amino acid sequence of AEFRHDS (SEQ ID NO:12).

18. A peptide comprising the structure:
[first peptide]-[linker 1]-[second peptide]-[linker 2]-[Cys],
wherein, the first peptide is a peptide according to claim 1, the second peptide is the same or different peptide according to claim 1 each of linker 1, linker 2 and [Cys] are optional, and linker 1 and linker 2 may be the same or different.

19. The peptide of claim 18, wherein the first peptide or the second peptide comprises 3-10 amino acids from residues 1-10 of SEQ ID NO:01.

20. The peptide of claim 18, wherein both the first peptide and the second peptide comprise 3-10 amino acids from residues 1-10 of SEQ ID NO:01.

21. The peptide of claim 18, wherein the first peptide or the second peptide comprises 3-10 amino acids from residues 12-25 of SEQ ID NO:01.

22. The peptide of claim 18, wherein both the first peptide and the second peptide comprise 3-10 amino acids from residues 12-25 of SEQ ID NO:01.

23. The peptide of claim 18, wherein the first peptide or the second peptide is selected from the group consisting of SEQ ID NO:02 through SEQ ID NO:39.

24. The peptide of claim 18, wherein both the first peptide and the second peptide are selected from the group consisting of SEQ ID NO:02 through SEQ ID NO:39.

25. The peptide of claim 18, wherein the first peptide or the second peptide is selected from the group consisting of SEQ ID NO:40 through SEQ ID NO:96.

26. The peptide of claim 18, wherein both the first peptide and the second peptide are selected from the group consisting of SEQ ID NO:40 through SEQ ID NO:96.

27. The peptide of claim 18, wherein the first peptide or the second peptide comprises 3-10 amino acids from residues 1-10 of SEQ ID NO:01, and the other peptide comprises 3-10 amino acids from residues 12-25 of SEQ ID NO:01.

28. The peptide of claim 18, wherein the first peptide or the second peptide is selected from the group consisting of SEQ ID NO:02 through SEQ ID NO:39, and the other peptide is selected from the group consisting of SEQ ID NO:40 through SEQ ID NO:96.

29. The peptide of claim 18, wherein the first peptide or the second peptide is an amino acid sequence selected from the group consisting of SEQ ID NO:02 through SEQ ID NO:96.

30. The peptide of claim 18, wherein both of the first peptide and the second peptide is an amino acid sequence selected from the group consisting of SEQ ID NO:02 through SEQ ID NO:96.

31. The peptide of claim 18, wherein at least one of the first peptide or the second peptide is selected from the group consisting of the peptides of claim 8.

32. The peptide of claim 18, wherein both the first peptide and the second peptide are selected from the group consisting of the peptides of claim 8.

33. The peptide of claim 18, wherein the first peptide or the second peptide is SEQ ID NO:101.

34. The peptide of claim 18, wherein both the first peptide and the second peptide are SEQ ID NO:101.

35. The peptide of claim 18, wherein the first peptide and the second peptide are each selected from the group consisting of SEQ ID NO:02 through SEQ ID NO:96, and SEQ ID NO:101.

36. The peptide of any of claims 1 to 8, further comprising a linker at a C-terminal portion of the peptide.

37. The peptide of claim 36, wherein the linker comprises an amino acid sequence.

38. The peptide of claim 37, wherein the linker comprises an amino acid sequence selected from the group consisting of AA, AAA, KK, KKK, SS, SSS, AGAG (SEQ ID NO:99), GG, GGG, GAGA (SEQ ID NO:98), and KGKG (SEQ ID NO:100).

39. The peptide of claim 38, wherein the linker further comprises a C-terminal cysteine (C).

40. The peptide of claim 38, wherein the peptide further comprises a blocked amine at the N-terminus.

41. The peptide of claim 18, wherein the first linker, is a cleavable linker.

42. An immunotherapy composition, comprising one or more of the peptides of any of claims 1 to 41.

43. The immunotherapy composition of claim 42, wherein the one or more peptides further comprises a linker to a carrier at a C-terminal portion of the peptide.

44. The immunotherapy composition of claim 43, wherein the linker comprises an amino acid sequence selected from the group consisting of AA, AAA, KK, KKK, SS, SSS, AGAG (SEQ ID NO:99), GG, GGG, GAGA (SEQ ID NO:98), and KGKG (SEQ ID NO:100).

45. The immunotherapy composition of either of claim 43 or 44, wherein the carrier comprises serum albumins, immunoglobulin molecules, thyroglobulin, ovalbumin, tetanus toxoid (TT), diphtheria toxoid (DT), a genetically modified cross-reacting material (CRM) of diphtheria toxin, CRM197, meningococcal outer membrane protein complex (OMPC) and H. influenzae protein D (HiD), rEPA (Pseudomonas aeruginosa exotoxin A), KLH (keyhole limpet hemocyanin), and flagellin.

46. The immunotherapy composition of claim 45, wherein the carrier is CRM 197.

47. The immunotherapy composition of claim 45, wherein the carrier is diphtheria toxoid.

48. The immunotherapy composition of any one of claims 42-47, further comprising at least one pharmaceutically acceptable diluent.

49. The immunotherapy composition of any one of claims 42-47, further comprising a multiple antigen presenting system (MAP).

50. The immunotherapy composition of claim 49, wherein the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

51. A pharmaceutical composition comprising (a) one or more of the polypeptide of any one of claims 1 to 41 or (b) the immunotherapy composition of any of claims 42 to 50 and at least one adjuvant.

52. The pharmaceutical composition of claim 51, wherein the adjuvant is selected from the group consisting of aluminum hydroxide, aluminum phosphate, aluminum sulfate, 3 De-O-acylated monophosphoryl lipid A (MPL), QS-21, TQL1055, QS-18, QS-17, QS-7, Complete Freund’s Adjuvant (CFA), Incomplete Freund’s Adjuvant (IFA), oil in water emulsions (such as squalene or peanut oil), CpG, polyglutamic acid, polylysine, AddaVax™, MF59®, and combinations thereof.

53. The pharmaceutical composition of claim 52, wherein the adjuvant is QS-21 or TQL1055.

54. The pharmaceutical composition of claim 52, wherein the adjuvant is MPL.

55. The pharmaceutical composition of claim 52, wherein the adjuvant is a combination of MPL and QS-21 or a combination of MPL and TQL1055.

56. The pharmaceutical composition of any of claims 51 to 55, wherein the adjuvant comprises a liposomal formulation.

57. The pharmaceutical composition of any of claims 51 to 56, wherein the composition comprises at least one pharmaceutically acceptable diluent.

58. The pharmaceutical composition of any of claims 51 to 56, comprising a multiple antigen presenting system (MAP).

59. The pharmaceutical composition of claim 58, wherein the MAP comprises one or more of a Lys-based dendritic scaffold, helper T-cell epitopes, immune stimulating lipophilic moieties, cell penetrating peptides, radical induced polymerization, self-assembling nanoparticles as antigen-presenting platforms and gold nanoparticles.

60. A nucleic acid comprising a nucleic acid sequence encoding a peptide of any one of claims 1 to 41 or the immunotherapy composition of claims 42 to 50.

61. A nucleic acid immunotherapy composition comprising the nucleic acid of claim 60 and at least one adjuvant.

62. A method of treating or effecting prophylaxis of Alzheimer’s disease in a subject, comprising administrating to the subject the immunotherapy composition of any of claims 42-50 or the pharmaceutical compositions of any of claims 51 to 59.

63. A method of inhibiting or reducing aggregation of Aβ in a subject having or at risk of developing Alzheimer’s disease, comprising, administering to the subject the immunotherapy composition of any of claims 42 to 50 or the pharmaceutical composition of any of claims 51 to 59.

64. A method of treating or effecting prophylaxis of Alzheimer’s disease in a subject, comprising administrating to the subject the nucleic acid immunotherapy composition of claim 60 or claim 61.

65. A method of inhibiting or reducing aggregation of Aβ in a subject having or at risk of developing Alzheimer’s disease, comprising administering to the subject the nucleic acid immunotherapy composition of claim 60 or claim 61.

66. The method of any of claims 62 to 65, further comprising repeating the administering at least a second time, at least a third time, at least a fourth time, at least a fifth time, or at least a sixth time.

67. The method of claim 66, further comprising repeating the administering at an interval of about 14 days, or about 21 to about 28 days, or about quarterly, or about biannually, or about annually.

68. A method of inducing an immune response in an animal, comprising administering to the animal any one of the polypeptide of claims 1 to 41, the immunotherapy composition of claims 42 to 50, the pharmaceutical compositions of claims 51 to 59 or the nucleic acid immunotherapy composition of claim 60 or claim 61 in a regimen effective to generate an immune response comprising antibodies that specifically bind to Aβ.

69. The method of claim 68, wherein the immune response comprises antibodies that specifically bind to Aβ.

70. The method of either of claims 68 or 69, wherein the inducing the immune response comprises antibodies that specifically bind to the N-terminal region of Aβ.

71. An immunization kit comprising the immunotherapy composition of any of claims 42 to 50.

72. The kit of claim 71, further comprising an adjuvant.

73. The kit of claim 72, wherein the immunotherapy composition is in a first container and the adjuvant is in a second container.

74. A kit comprising the nucleic acid immunotherapy composition of claim 60 or claim 61.

75. The kit of claim 74, further comprising an adjuvant.

76. The kit of claim 75, wherein the nucleic acid is in a first container and the adjuvant is in a second container.


SEQUENCE LISTING (101 sequences)

SEQ ID NO: 1 - protein, 42 aa, Artificial Sequence [sequence omitted]

SEQ ID NO: 2 - protein, 10 aa, Artificial Sequence
  Synthetic peptide
DAEFRHDSGY

SEQ ID NO: 3 - protein, 9 aa, Artificial Sequence
  Synthetic peptide
DAEFRHDSG

SEQ ID NO: 4 - protein, 8 aa, Artificial Sequence
  Synthetic peptide
DAEFRHDS

SEQ ID NO: 5 - protein, 7 aa, Artificial Sequence
  Synthetic peptide
DAEFRHD

SEQ ID NO: 6 - protein, 6 aa, Artificial Sequence
  Synthetic peptide
DAEFRH

SEQ ID NO: 7 - protein, 5 aa, Artificial Sequence
  Synthetic peptide
DAEFR

SEQ ID NO: 8 - protein, 4 aa, Artificial Sequence
  Synthetic peptide
DAEF

SEQ ID NO: 9 - protein, 3 aa, Artificial Sequence
  Synthetic peptide
DAE

SEQ ID NO: 10 - protein, 9 aa, Artificial Sequence
  Synthetic peptide
AEFRHDSGY

SEQ ID NO: 11 - protein, 8 aa, Artificial Sequence
  Synthetic peptide
AEFRHDSG

SEQ ID NO: 12 - protein, 7 aa, Artificial Sequence
  Synthetic peptide
AEFRHDS

SEQ ID NO: 13 - protein, 6 aa, Artificial Sequence
  Synthetic peptide
AEFRHD

SEQ ID NO: 14 - protein, 5 aa, Artificial Sequence
  Synthetic peptide
AEFRH

SEQ ID NO: 15 - protein, 4 aa, Artificial Sequence
  Synthetic peptide
AEFR

SEQ ID NO: 16 - protein, 3 aa, Artificial Sequence
  Synthetic peptide
AEF

SEQ ID NO: 17 - protein, 8 aa, Artificial Sequence
  Synthetic peptide
EFRHDSGY

SEQ ID NO: 18 - protein, 7 aa, Artificial Sequence
  Synthetic peptide
EFRHDSG

SEQ ID NO: 19 - protein, 6 aa, Artificial Sequence
  Synthetic peptide
EFRHDS

SEQ ID NO: 20 - protein, 5 aa, Artificial Sequence
  Synthetic peptide
EFRHD

SEQ ID NO: 21 - protein, 4 aa, Artificial Sequence
  Synthetic peptide
EFRH

SEQ ID NO: 22 - protein, 3 aa, Artificial Sequence
  Synthetic peptide
EFR

SEQ ID NO: 23 - protein, 7 aa, Artificial Sequence
  Synthetic peptide
FRHDSGY

SEQ ID NO: 24 - protein, 6 aa, Artificial Sequence
  Synthetic peptide
FRHDSG

SEQ ID NO: 25 - protein, 5 aa, Artificial Sequence
  Synthetic peptide
FRHDS

SEQ ID NO: 26 - protein, 4 aa, Artificial Sequence
  Synthetic peptide
FRHD

SEQ ID NO: 27 - protein, 3 aa, Artificial Sequence
  Synthetic peptide
FRH

SEQ ID NO: 28 - protein, 6 aa, Artificial Sequence
  Synthetic peptide
RHDSGY

SEQ ID NO: 29 - protein, 5 aa, Artificial Sequence
  Synthetic peptide
RHDSG

SEQ ID NO: 30 - protein, 4 aa, Artificial Sequence
  Synthetic peptide
RHDS

SEQ ID NO: 31 - protein, 3 aa, Artificial Sequence
  Synthetic peptide
RHD

SEQ ID NO: 32 - protein, 5 aa, Artificial Sequence
  Synthetic peptide
HDSGY

SEQ ID NO: 33 - protein, 4 aa, Artificial Sequence
  Synthetic peptide
HDSG

SEQ ID NO: 34 - protein, 3 aa, Artificial Sequence
  Synthetic peptide
HDS

SEQ ID NO: 35 - protein, 4 aa, Artificial Sequence
  Synthetic peptide
DSGY

SEQ ID NO: 36 - protein, 3 aa, Artificial Sequence
  Synthetic peptide
DSG

SEQ ID NO: 37 - protein, 3 aa, Artificial Sequence
  Synthetic peptide
SGY

SEQ ID NO: 38 - protein, 9 aa, Artificial Sequence
  Synthetic peptide
AEFRHDSGC

SEQ ID NO: 39 - protein, 8 aa, Artificial Sequence
  Synthetic peptide
DAEFRHDC

SEQ ID NO: 40 - protein, 9 aa, Artificial Sequence
  Synthetic peptide
QKLVFFAEC

SEQ ID NO: 41 - protein, 10 aa, Artificial Sequence
  Synthetic peptide
VHHQKLVFFA

SEQ ID NO: 42 - protein, 9 aa, Artificial Sequence
  Synthetic peptide
VHHQKLVFF

SEQ ID NO: 43 - protein, 8 aa, Artificial Sequence
  Synthetic peptide
VHHQKLVF

SEQ ID NO: 44 - protein, 7 aa, Artificial Sequence
  Synthetic peptide
VHHQKLV

SEQ ID NO: 45 - protein, 6 aa, Artificial Sequence
  Synthetic peptide
VHHQKL

SEQ ID NO: 46 - protein, 10 aa, Artificial Sequence
  Synthetic peptide
HHQKLVFFAE

SEQ ID NO: 47 - protein, 9 aa, Artificial Sequence
  Synthetic peptide
HHQKLVFFA

SEQ ID NO: 48 - protein, 8 aa, Artificial Sequence
  Synthetic peptide
HHQKLVFF

SEQ ID NO: 49 - protein, 7 aa, Artificial Sequence
  Synthetic peptide
HHQKLVF

SEQ ID NO: 50 - protein, 6 aa, Artificial Sequence
  Synthetic peptide
HHQKLV

SEQ ID NO: 51 - protein, 5 aa, Artificial Sequence
  Synthetic peptide
HHQKL

SEQ ID NO: 52 - protein, 10 aa, Artificial Sequence
  Synthetic peptide
HQKLVFFAED

SEQ ID NO: 53 - protein, 9 aa, Artificial Sequence
  Synthetic peptide
HQKLVFFAE

SEQ ID NO: 54 - protein, 8 aa, Artificial Sequence
  Synthetic peptide
HQKLVFFA

SEQ ID NO: 55 - protein, 7 aa, Artificial Sequence
  Synthetic peptide
HQKLVFF

SEQ ID NO: 56 - protein, 6 aa, Artificial Sequence
  Synthetic peptide
HQKLVF

SEQ ID NO: 57 - protein, 5 aa, Artificial Sequence
  Synthetic peptide
HQKLV

SEQ ID NO: 58 - protein, 4 aa, Artificial Sequence
  Synthetic peptide
HQKL

SEQ ID NO: 59 - protein, 10 aa, Artificial Sequence
  Synthetic peptide
QKLVFFAEDV

SEQ ID NO: 60 - protein, 9 aa, Artificial Sequence
  Synthetic peptide
QKLVFFAED

SEQ ID NO: 61 - protein, 8 aa, Artificial Sequence
  Synthetic peptide
QKLVFFAE

SEQ ID NO: 62 - protein, 7 aa, Artificial Sequence
  Synthetic peptide
QKLVFFA

SEQ ID NO: 63 - protein, 6 aa, Artificial Sequence
  Synthetic peptide
QKLVFF

SEQ ID NO: 64 - protein, 5 aa, Artificial Sequence
  Synthetic peptide
QKLVF

SEQ ID NO: 65 - protein, 4 aa, Artificial Sequence
  Synthetic peptide
QKLV

SEQ ID NO: 66 - protein, 3 aa, Artificial Sequence
  Synthetic peptide
QKL

SEQ ID NO: 67 - protein, 10 aa, Artificial Sequence
  Synthetic peptide
KLVFFAEDVG

SEQ ID NO: 68 - protein, 9 aa, Artificial Sequence
  Synthetic peptide
KLVFFAEDV

SEQ ID NO: 69 - protein, 8 aa, Artificial Sequence
  Synthetic peptide
KLVFFAED

SEQ ID NO: 70 - protein, 7 aa, Artificial Sequence
  Synthetic peptide
KLVFFAE

SEQ ID NO: 71 - protein, 6 aa, Artificial Sequence
  Synthetic peptide
KLVFFA

SEQ ID NO: 72 - protein, 5 aa, Artificial Sequence
  Synthetic peptide
KLVFF

SEQ ID NO: 73 - protein, 4 aa, Artificial Sequence
  Synthetic peptide
KLVF

SEQ ID NO: 74 - protein, 3 aa, Artificial Sequence
  Synthetic peptide
KLV

SEQ ID NO: 75 - protein, 9 aa, Artificial Sequence
  Synthetic peptide
LVFFAEDVG

SEQ ID NO: 76 - protein, 8 aa, Artificial Sequence
  Synthetic peptide
LVFFAEDV

SEQ ID NO: 77 - protein, 7 aa, Artificial Sequence
  Synthetic peptide
LVFFAED

SEQ ID NO: 78 - protein, 6 aa, Artificial Sequence
  Synthetic peptide
LVFFAE

SEQ ID NO: 79 - protein, 5 aa, Artificial Sequence
  Synthetic peptide
LVFFA

SEQ ID NO: 80 - protein, 4 aa, Artificial Sequence
  Synthetic peptide
LVFF

SEQ ID NO: 81 - protein, 3 aa, Artificial Sequence
  Synthetic peptide
LVF

SEQ ID NO: 82 - protein, 8 aa, Artificial Sequence
  Synthetic peptide
VFFAEDVG

SEQ ID NO: 83 - protein, 7 aa, Artificial Sequence
  Synthetic peptide
VFFAEDV

SEQ ID NO: 84 - protein, 6 aa, Artificial Sequence
  Synthetic peptide
VFFAED

SEQ ID NO: 85 - protein, 5 aa, Artificial Sequence
  Synthetic peptide
VFFAE

SEQ ID NO: 86 - protein, 4 aa, Artificial Sequence
  Synthetic peptide
VFFA

SEQ ID NO: 87 - protein, 3 aa, Artificial Sequence
  Synthetic peptide
VFF

SEQ ID NO: 88 - protein, 7 aa, Artificial Sequence
  Synthetic peptide
FFAEDVG

SEQ ID NO: 89 - protein, 6 aa, Artificial Sequence
  Synthetic peptide
FFAEDV

SEQ ID NO: 90 - protein, 5 aa, Artificial Sequence
  Synthetic peptide
FFAED

SEQ ID NO: 91 - protein, 4 aa, Artificial Sequence
  Synthetic peptide
FFAE

SEQ ID NO: 92 - protein, 3 aa, Artificial Sequence
  Synthetic peptide
FFA

SEQ ID NO: 93 - protein, 6 aa, Artificial Sequence
  Synthetic peptide
FAEDVG

SEQ ID NO: 94 - protein, 5 aa, Artificial Sequence
  Synthetic peptide
FAEDV

SEQ ID NO: 95 - protein, 4 aa, Artificial Sequence
  Synthetic peptide
FAED

SEQ ID NO: 96 - protein, 3 aa, Artificial Sequence
  Synthetic peptide
FAE

SEQ ID NO: 97 - protein, 4 aa, Artificial Sequence
  Synthetic peptide
RVRR

SEQ ID NO: 98 - protein, 4 aa, Artificial Sequence
  Synthetic peptide
GAGA

SEQ ID NO: 99 - protein, 4 aa, Artificial Sequence
  Synthetic peptide
AGAG

SEQ ID NO: 100 - protein, 4 aa, Artificial Sequence
  Synthetic peptide
KGKG

SEQ ID NO: 101 - protein, 9 aa, Artificial Sequence
  Synthetic peptide
DAEFRHDRR